(19) United States
(12) Patent Application Publication
(10) Pub. No.: US 2024/0065667 A1
(43) Pub. Date: Feb. 29, 2024

(54) Methods, Systems, Devices and Components for Rapid On-Site Measurement, Characterization and Classification of a Patient's Carotid Artery Using a Portable Ultrasound Probe or Device

(71) Applicant: Sightin Health SA, Crans-Montana (CH)

(72) Inventors: Marius Jatautas, Geneva (CH); Andreu Romero Arderiu, Lausanne (CH); Pablo Cañas Castellanos, Lausanne (CH); Mehdi Namdar, Crans (CH); William Frederick King, IV, Cologny (CH)

(21) Appl. No.: 18/238,993

(22) Filed: Aug. 28, 2023

Related U.S. Application Data
(60) Provisional application No. 63/401,387, filed on Aug. 26, 2022.

(51) Int. Cl. A61B 8/08; A61B 8/00

(52) U.S. Cl. CPC A61B 8/085; A61B 8/0891; A61B 8/4427; A61B 8/461; A61B 8/5207

(57) ABSTRACT

Described and disclosed herein are various embodiments of methods and systems configured to rapidly measure, analyze and provide in, for example, an on-site and out-patient setting within a short period of time, one or more physical parameters associated with a one or more of a patient's carotid arteries. Some embodiments comprise at least one computing device, at least one portable handheld ultrasound device or probe operably connected to the at least one computing device, the portable handheld ultrasound device being configured to provide to the computing device as outputs therefrom a series of ultrasound image frames acquired from the one or more of the patient's carotid arteries, and a display or monitor operably connected to the at least one computing device and configured to visually display to a user results generated by the at least one computing device. Among other things, the ultrasound image frames are processed to guide a clinician in accurately, quickly and efficiently placing the ultrasound probe on the patient's neck and body so that the physical characteristics of a patient's carotid arteries can be accurately detected and measured.

### RELATED APPLICATIONS

This application claims priority and other benefits from U.S. Provisional Patent Application Ser. No. 63/401,387 to Jatautas et al. filed on Aug. 26, 2022 and entitled Methods, Systems, Devices and Components for Rapid On-Site Measurement, Characterization and Classification of a Patient's Carotid Artery Using a Portable Ultrasound Probe or Device” (hereafter “the '387 patent application”). The '387 patent application is hereby incorporated by reference herein, in its entirety.

### FIELD OF THE INVENTION

The various embodiments described and disclosed herein include those relating to systems, devices, components and methods for rapid on-site characterization and classification of a patient's carotid artery, which in some embodiments comprises processing brightness mode ultrasound images in medical applications for the measurement of cardiovascular parameters.

### BACKGROUND

Carotid intima-media thickness (cIMT) testing via brightness mode (B-mode) ultrasound is a known method for evaluating cardiovascular risk by measuring the thickness of the intimal and medial layers of the artery wall. It advantageously allows non-invasive ultrasonic detection of carotid plaque and cIMT measurements that may be used to assess cardiovascular risks in a patient and detect possible vascular disease. The medical practitioner typically uses a portable ultrasound device to scan a patient's carotid artery, and based on the captured images, may perform a diagnosis including a measurement of the cIMT, plaque localization, plaque volume, maximum plaque height, plaque echogenicity, intraplaque neovascularization, stenosis progression and arterial stiffness in combination with a blood measurement device. These various assessments may be used to evaluate an individual's cardiovascular risk.

Accurate and reliable assessment of pictorial information by a skilled sonographer may vary depending on multiple factors including the level of skill of the sonographer, the morphology of the patient, and the precision of the instruments used. There may be quite a large variability in the diagnosis based on the pictorial assessments made by the practitioner. There would therefore be need to provide a more reliable diagnosis and a system to assist the practitioner in performing the assessment and reading the results.

### SUMMARY

Various embodiments provide a system for evaluating cardiovascular health parameters using portable brightness mode ultrasound devices that generates reliable and reproduceable results and that assists the healthcare practitioner in performing an accurate diagnosis.

According to one embodiment, there is provided a system configured to rapidly measure, analyze and provide, in an on-site or out-patient setting and within a predetermined period of time, one or more physical parameters or characteristics associated with a one or more of a patient's carotid arteries, where the system comprises at least one computing device; at least one portable ultrasound device or probe operably connected to the at least one computing device, the portable ultrasound device being configured to provide to the computing device as outputs therefrom a series of ultrasound image frames acquired from the one or more of the patient's carotid arteries, and a display or monitor operably connected to the at least one computing device and configured to visually display to a user results generated by the at least one computing device; wherein the computing device comprises at least one non-transitory computer readable medium configured to store instructions executable by at least one processor to process the ultrasound image frames, the at least one processor and the at least one non-transitory computer readable medium further being configured to process the ultrasound image frames using a trained discriminative or convolutional machine learning model, the computing device and ultrasound device or probe being configured to: (i) identify at least one of a bifurcation, a transverse view, and a cross-sectional view of at least one of the patient's carotid arteries from among the ultrasound image frames generated by the ultrasound device or probe; (ii) generate directional and locational instructions to the user on the display or monitor or via sound regarding the location, placement, orientation and movement of the ultrasound device or probe; (iii) measure and compute at least one of lumen distention, carotid intima-media thickness (cIMT), cardiovascular age, plaque characterization, and local arterial stiffness of the at least one carotid artery; and (iv) provide as outputs from the computing device at least one of the measured and computed lumen distention, the measured and computed carotid intima-media thickness (cIMT), the measured and computed cardiovascular age, the measured and computed plaque characterization, and the measured and computed local arterial stiffness of the at least one carotid artery to one or more of the monitor or display, a printer, a speaker or headphones, or data formatted for digital or memory storage or transmission.

Such an embodiment may further comprise one or more of: (a) the ultrasound device or probe being configured to switchably and controllably operate in B-mode or M-mode; (b) the ultrasound device or probe being configured to switchably and controllably operate in the M-mode when measuring lumen distension; (c) the trained discriminative or convolutional machine learning model being trained and configured to generate directional and locational instructions to the user on the display or monitor or via sound regarding at least one of the location, placement, orientation and movement of the ultrasound device or probe to identify one or more of carotid artery bifurcation locations, carotid artery cross-sectional characteristics, carotid artery plaque characteristics, carotid artery lumen characteristics, carotid artery regions of interest, and carotid artery far wall variability; (d) the trained discriminative or convolutional machine learning model is trained and configured to provide directional and locational instructions to the user regarding whether the ultrasound probe or device is centered over the patient's carotid artery; (e) lumen distension being measured and computed using the patient's blood pressure as an input; (f) the trained discriminative or convolutional machine learning model being trained and configured to crop the ultrasound image frames so as to enhance reliability of the detection and identification one or more of carotid artery bifurcation locations, carotid artery cross-sectional characteristics, carotid artery plaque characteristics, carotid artery lumen characteristics, carotid artery regions of interest, and carotid artery far wall variability; (g) the trained discriminative or convolutional machine learning model being trained and configured to determine the quality of the ultrasound image frames so as to enhance reliability of the detection and identification one or more of carotid artery bifurcation locations, carotid artery cross-sectional characteristics, carotid artery plaque characteristics, carotid artery lumen characteristics, carotid artery regions of interest, and carotid artery far wall variability; (h) the trained discriminative or convolutional machine learning model being trained and configured to measure and compute one or more of the number, location, length, height, area and echogenicity of each plaque in the at least one carotid artery as part of the plaque characterization; and (i) the predetermined period of time being about ten minutes or less.

In another embodiment, there is provided a method of rapidly measuring, analyzing and providing, in an on-site and out-patient setting within a predetermined period of time, one or more physical parameters associated with a one or more of a patient's carotid arteries, the system comprising at least one computing device, at least one portable handheld ultrasound device or probe operably connected to the at least one computing device, the portable handheld ultrasound device being configured to provide to the computing device as outputs therefrom a series of ultrasound image frames acquired from one or more of the patient's carotid arteries, and a display or monitor operably connected to the at least one computing device and configured to visually display to a user results generated by the at least one computing device, the computing device comprising at least one non-transitory computer readable medium configured to store instructions executable by at least one processor to process the ultrasound image frames, the at least one processor and the at least one non-transitory computer readable medium further being configured to process the ultrasound image frames using a trained discriminative or convolutional machine learning model, using the computing device, ultrasound device and the display or monitor, the method comprising: (i) identifying at least one of a bifurcation, a transverse view, and a cross-sectional view of at least one of the patient's carotid arteries from among the ultrasound image frames generated by the ultrasound device or probe; (ii) generating directional and locational instructions to the user on the display or monitor or via sound regarding the location, placement, orientation and movement of the ultrasound device or probe; (iii) measuring and computing at least one of lumen distention, carotid intima-media thickness (cIMT), cardiovascular age, plaque characterization, and local arterial stiffness of the at least one carotid artery; and (iv) providing as outputs from the computing device at least one of the measured and computed lumen distention, the measured and computed carotid intima-media thickness (cIMT), the measured and computed cardiovascular age, the measured and computed plaque characterization, and the measured and computed local arterial stiffness of the at least one carotid artery to one or more of the monitor or display, a printer, a speaker or headphones, or data formatted for digital or memory storage or transmission.

Such an embodiment may further comprise one or more of: (a) the ultrasound device or probe being configured to switchably and controllably operate in B-mode or M-mode; (b) the ultrasound device or probe being configured to switchably and controllably operate in the M-mode when measuring lumen distension; (c) the trained discriminative or convolutional machine learning model being trained and configured to generate directional and locational instructions to the user on the display or monitor or via sound regarding at least one of the location, placement, orientation and movement of the ultrasound device or probe to identify one or more of carotid artery bifurcation locations, carotid artery cross-sectional characteristics, carotid artery plaque characteristics, carotid artery lumen characteristics, carotid artery regions of interest, and carotid artery far wall variability; (d) the trained discriminative or convolutional machine learning model is trained and configured to provide directional and locational instructions to the user regarding whether the ultrasound probe or device is centered over the patient's carotid artery; (e) lumen distension being measured and computed using the patient's blood pressure as an input; (f) the trained discriminative or convolutional machine learning model being trained and configured to crop the ultrasound image frames so as to enhance reliability of the detection and identification one or more of carotid artery bifurcation locations, carotid artery cross-sectional characteristics, carotid artery plaque characteristics, carotid artery lumen characteristics, carotid artery regions of interest, and carotid artery far wall variability; (g) the trained discriminative or convolutional machine learning model being trained and configured to determine the quality of the ultrasound image frames so as to enhance reliability of the detection and identification one or more of carotid artery bifurcation locations, carotid artery cross-sectional characteristics, carotid artery plaque characteristics, carotid artery lumen characteristics, carotid artery regions of interest, and carotid artery far wall variability; (h) the trained discriminative or convolutional machine learning model being trained and configured to measure and compute one or more of the number, location, length, height, area and echogenicity of each plaque in the at least one carotid artery as part of the plaque characterization; and (i) the predetermined period of time being about ten minutes or less.

In still another embodiment, there is provided a system for processing brightness mode ultrasound images in medical applications, in particular for measurement of cardiovascular parameters, comprising a computing system and program modules executable in the computing system configured to process data generated by a B-mode ultrasound probe device of a patient's carotid artery, the program modules include modules for processing an image generated by the B-mode ultrasound probe device, including automated frame cropping of the image, lumen delineation, far wall ROI segmentation and cIMT delineation, wherein the lumen delineation module is configured to: divide the image in a plurality of columns extending from a first side of the image captured closest to the ultrasound probe device to a second opposite side of the image captured furthest from the ultrasound probe device; determine in each column at least a first and a last significant local intensity maxima I1, I2, I3 of image pixels above a chosen fixed intensity threshold; determine in each column at least one significant local intensity minima i1, i2 of image pixels between the first significant local intensity maxima I1 and the last significant local intensity maxima I3; compute paths of connected pixels from the local significant local intensity minima i1, i2 within a predefined distance from the significant local intensity maxima; calculate the lengths of the paths; select paths having a length greater than a fixed proportion of a path with the greatest length, the fixed proportion in a range of 0.7 to 0.95, and define a lumen axis as the selected path furthest from the first side of the image.

Such an embodiment may further comprise one or more of: (a) the intensity value of image pixels being normalized in a fixed scale, for example from 0 to 1; (b) fixed intensity threshold being in a range of 0.1 to 0.3 of the fixed scale, preferably in a range of 0.15 to 0.25; (c) three the significant local intensity maxima I1, I2, I3 of image pixels above a chosen fixed intensity threshold being determined, and two of the significant local intensity minima i1, i2 being determined; (d) the predefined distance from the significant local intensity maxima falls within a predetermined range; (e) the frame cropping module being configured to generate a binary mask in which pixels whose intensity values lie between a first intensity threshold T1 and a second intensity threshold T2 are set to bright, and pixels having an intensity below the first threshold and above the second threshold are set to dark, and wherein the frame cropping or lumen delineation module is configured to apply a soothing filter, for example a gaussian smoothing filter, to the binary mask for further processing by the lumen delineation module; (f) the far wall ROI segmentation and cIMT delineation modules being configured to generate a binary mask of the image after the lumen delineation processing in which a threshold T is computed to separate pixels into two classes according to their brightness intensity: pixels having intensities below the threshold set to dark pixels and corresponding to the lumen; pixels having intensities above the threshold set to bright and corresponding to the wall interfaces below the lumen and other hyperechoic tissues; and identify a far wall line of the artery in columns extending from the first side of the image to the second opposite side of the image, as the first line of bright pixels extending laterally across the columns; (g) the far wall ROI segmentation and cIMT delineation modules being configured to smooth the far wall line with a smoothing filter (for example a Savitzky-Golay filter), and define upper and lower bounds of a far wall ROI with a predefined height parameter to generate a far wall ROI image; (h) the cIMT delineation module is configured to apply a filter (for example a gaussian filter) to the far wall ROI image to reduce speckle noise and generate a smoothed intensity map, apply a Sobel filter to the smoothed intensity map in the column direction y to retrieve a y-gradient map of a region of interest, retrieve continuous paths passing through maximum intensity values defined as a center of adventitia layer, retrieve all gradient local maxima located above the adventitia center, select a pre-defined number of the longest paths and retaining overlapping paths considered as or determined to be lumen-intima or media-adventitia candidates, the paths located closest to the first side being identified as lumen-intima, and the paths closest to the second side being identified as the media-adventitia, define as ROI a section having a pair of paths with the largest overlap; and (i) the program modules comprise a transverse carotid and bifurcation identification module to provide visual feedback to assist a medical practitioner in identifying a carotid artery and a bifurcation of the carotid artery, the transverse carotid and bifurcation identification module configured to identify and mark with a bounding box the carotid in a transverse B-mode ultrasound image, and identify the bifurcation if a ratio of a length to height of the bounding box is above a certain fixed threshold.

In yet another embodiment, there is provided a method of processing brightness mode ultrasound images in medical applications, in particular for measurement of cardiovascular parameters, comprising executing program modules in a computing system configured to process data generated by a B-mode ultrasound probe device of a patient's carotid artery, the program modules include modules for processing an image generated by the B-mode ultrasound probe device, including automated frame cropping of the image, lumen delineation, far wall ROI segmentation and cIMT delineation, wherein the lumen delineation module performs the following steps: divide the image in a plurality of columns extending from a first side of the image captured closest to the ultrasound probe device to a second opposite side of the image captured furthest from the ultrasound probe device; determine in each column at least a first and a last significant local intensity maxima I1, I2, I3 of image pixels above a chosen fixed intensity threshold, determine in each column at least one significant local intensity minima i1, i2 of image pixels between the first significant local intensity maxima I1 and the last significant local intensity maxima I3, compute paths of connected pixels from the local significant local intensity minima i1, i2 within a predefined distance from the significant local intensity maxima, calculate the lengths of the paths, select paths having a length greater than a fixed proportion of a path with the greatest length, the fixed proportion being in a range of 0.7 to 0.95, and define a lumen axis as the I1 selected path furthest from the first side of the image.

Such an embodiment may further comprise one or more of: (a) the intensity value of image pixels being normalized on a fixed scale, for example from 0 to 1 and the fixed intensity threshold being in a range of 0.1 to 0.3 of the fixed scale, preferably in a range of between about 0.15 and about 0.25; (b) three being significant local intensity maxima I1, I2, I3 of image pixels above a chosen fixed intensity threshold being determined, and two of the significant local intensity minima i1, i2 being determined; (c) the predefined distance from the significant local intensity maxima falls within a it predetermined range; (d) the frame cropping module generates a binary mask in which pixels whose intensity values lie between a first intensity threshold T1 and a second intensity threshold T2 are set to bright, and pixels having an intensity below the first threshold and above the second threshold are set to dark, and the frame cropping or lumen delineation module applies a soothing filter (for example a gaussian smoothing filter) to the binary mask for further processing by the lumen delineation module: (d) the far wall ROI segmentation and cIMT delineation modules generate a binary mask of the image after the lumen delineation processing in which a threshold T is computed to separate pixels into two classes according to their brightness intensity: pixels having intensities below the threshold set to dark pixels and corresponding to the lumen, pixels having intensities above the threshold set to bright and corresponding to the wall interfaces below the lumen and other hyperechoic tissues; identify a far wall line of the artery in columns extending from the first side of the image to the second opposite side of the image, as the first line of bright pixels extending laterally across the columns. (e) the far wall ROI segmentation and cIMT delineation modules smooth the far wall line with a smoothing filter (for example a Savitzky-Golay filter), and define upper and lower bounds of a far wall ROI with a predefined height parameter to generate a far wall ROI image; and (f) the cIMT delineation module applies a filter (for example a gaussian filter) to the far wall ROI image to reduce speckle noise and generate a smoothed intensity map, applies a Sobel filter on the smoothed intensity map in the column direction y to retrieve a y-gradient map of a region of interest, retrieves continuous paths passing through maximum intensity values defined as a center of adventitia layer, retrieves all gradient local maxima located above the adventitia center, selects a pre-defined number of the longest paths and retaining overlapping paths, which are considered as lumen-intima or media-adventitia candidates, the paths being located closest to the first side being identified as lumen-intima, and the paths closest to the second side being identified as the media-adventitia, and defines as and ROI a section having a pair of paths with the largest overlap.

It is advantageous to provide a system for processing images from brightness mode ultrasound devices that allows an accurate assessment of various cardiovascular health parameters including cIMT, plaque localization and measurement, and arterial stiffness.

It is advantageous to provide a system for assessment of cardiovascular health parameters using brightness mode ultrasound images which is cost efficient.

It is advantageous to provide a system for assessment of cardiovascular health parameters using brightness mode ultrasound images which allows to provide rapid and reliable measurements.

It is advantageous to provide a system for assessment of cardiovascular health parameters using brightness mode ultrasound images which facilitates the practitioners capture of ultrasound images in a more accurate manner.

Various embodiments have been achieved by providing a system for evaluating cardiovascular health parameters using portable brightness mode ultrasound devices according to the independent claims. Dependent claims set forth advantageous embodiments.

Further advantageous aspects of the various embodiments will be apparent from the claims, and from the following detailed description and accompanying Figures.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 is a schematic block diagram of a B-mode ultrasound image processing system for cardiovascular health assessment based on ultrasound images of a patient's carotid artery according to one embodiment;

FIG. 2A is a flow chart of an image capture process of the system of FIG. 1 according to one embodiment, in particular directed to the image capture of a transverse section of the carotid artery;

FIG. 2B is a flow chart of an image capture process of the system of FIG. 1 according to one embodiment, in particular directed an image capture of the carotid artery along a longitudinal axis;

FIG. 2C is a flow chart of a lumen and region of interest (ROI) detection process of the system of FIG. 1 according to one embodiment;

FIG. 2D is a flow chart of a cIMT and plaque characterization process of the system of FIG. 1 according to one embodiment;

FIG. 2E is a flow chart of a plaque characterization process according to one embodiment;

FIG. 3A is a pictorial illustration of the cardiovascular health parameters that a medical practitioner may assess based on B-mode ultrasound image capture of a patient;

FIG. 3B is a screenshot of an example of a measurement output of some health parameters of a patient using a system according to one embodiment;

FIGS. 4A through 12B Illustrate various images and plots illustrating specific examples of processes implemented according to some of the various embodiments;

FIG. 4A shows a short axis view of the common carotid artery, where the lumen is located inside the green bounding box;

FIG. 4B shows an image displaying the carotid bifurcation inside the red bounding box;

FIG. 5A shows an example of a raw B-mode ultrasound image;

FIG. 5B shows an example of a binary mask where white pixels correspond to pixels with intensities between the Otsu thresholds T1, T2;

FIG. 5C shows a binary mask after removing groups of isolated white pixels;

FIG. 6A shows an example of an ultrasound image of a longitudinal view of the carotid;

FIG. 6B shows an example raw image;

FIG. 6C shows an example gaussian-filtered image;

FIG. 6D shows a standardized intensity profile with local significant maxima flagged with red dots;

FIG. 6E shows a standardized intensity profile with lumen candidates flagged with green dots;

FIG. 6F shows a binary mask where 1's (blue dots) corresponds to lumen axis candidate positions;

FIG. 6G shows an image representing in different colors 3 different lumen axis paths candidates;

FIG. 6H shows a raw image with the final lumen axis choice displayed in green;

FIG. 7A shows a raw image with the lumen displayed in green;

FIG. 7B shows the resulting image after setting to 0 all pixels above the lumen;

FIG. 7C shows a binary mask where bright pixels correspond to all pixels whose intensity is above the Otsu threshold T1;

FIG. 7D shows a binary mask corresponding to the largest bright connected components;

FIG. 7E shows a binary mask with the first estimate of the far wall displayed in red;

FIG. 7F shows a binary mask with the final estimate of the far wall displayed in green;

FIG. 8A shows a far wall ROI raw image (left), and corresponding (Gaussian) smoothed intensity map (left) with the center of the estimated adventitia displayed as a red line;

FIG. 8B shows a y-gradient map of the far wall ROI obtained by applying a Sobel filter on the raw image;

FIG. 8C shows a binary mask where 1's (white pixels) corresponds to gradient local maxima in the y axis direction, located above the adventitia center. The adventitia center is displayed as a red continuous line;

FIG. 8D shows the largest 20 connected components that correspond to edges;

FIG. 8E shows final lumen-intima (red) and media-adventitia (yellow) candidates;

FIG. 8F shows final lumen-intima (red) and media-adventitia (yellow) delineations;

FIG. 9A shows a raw far wall ROI image (left) and a binary mask (right), where 1's (white pixels) correspond to gradient local maxima in the y axis direction, located above the adventitia center, and where the adventitia center is displayed as a red continuous line;

FIG. 9B shows a binary mask where 1's (bright pixels) represent strong edges;

FIG. 9C shows a binary mask where 1's (bright pixels) represent long and strong edges;

FIG. 9D: Initial lumen-intima delineation estimate (left), and raw far wall ROI image with the superposed lumen-intima delineation in red (right);

FIG. 9E: Binary mask result of thresholding the raw image with the threshold T*;

FIG. 9F: Largest connected component of the binary mask computed by thresholding the raw image with T*;

FIG. 9G: Largest connected component with the final lumen-intima delineation in red (left), and raw image with the final lumen-intima delineation in red (right);

FIG. 9H: Graph displaying the MSE against the different values of the threshold t. The MSE is minimized at T*=20;

FIG. 9I: y gradient map after applying a Sobel filter to the raw far wall ROI image;

FIG. 9J: Binary mask where 1's represents the lumen-intima basin. The red line represents the lumen-intima initial estimate;

FIG. 9K: Score map C(x,y);

FIG. 9L: Score map C(x,y) with the optimal path corresponding to the media-adventitia display as a red line;

FIG. 9M: Left: gradient profile of the media-adventitia pixels. In red a threshold of 0.2 times the global maxima is displayed. Right: raw image with the final media-adventitia delineation displayed in red;

FIG. 9N: Left: Final lumen-intima and media-adventitia delineations for plaque. Right Final smoothed lumen-intima and media-adventitia delineations for plaque;

FIG. 10: Image taken from Philipps QLab software, the QI=94 means that 94% of the points in the desired area were successfully delineated;

FIG. 11A: Far wall ROI image;

FIG. 11B: Gradient map of the far wall ROI image;

FIG. 11C: Gradient profile of a fixed column, with lumen-intima and media-adventitia points marked in red:

FIG. 12A: Far wall ROI raw image with the cIMT delineation in dark green and the plaque region located inside the light green bounding box;

FIG. 12B: Plaque composition according to pixel grayscale intensities, and

FIG. 13 shows a flow chart corresponding to another embodiment of an ultrasound image processing system for cardiovascular health assessment based on ultrasound images of a patient's carotid artery according to one embodiment, aspects of which are discussed and disclosed in the specification hereof

The drawings are not necessarily to scale. In general, like numbers refer to like parts or steps throughout the drawings.

### DETAILED DESCRIPTIONS OF SOME EMBODIMENTS

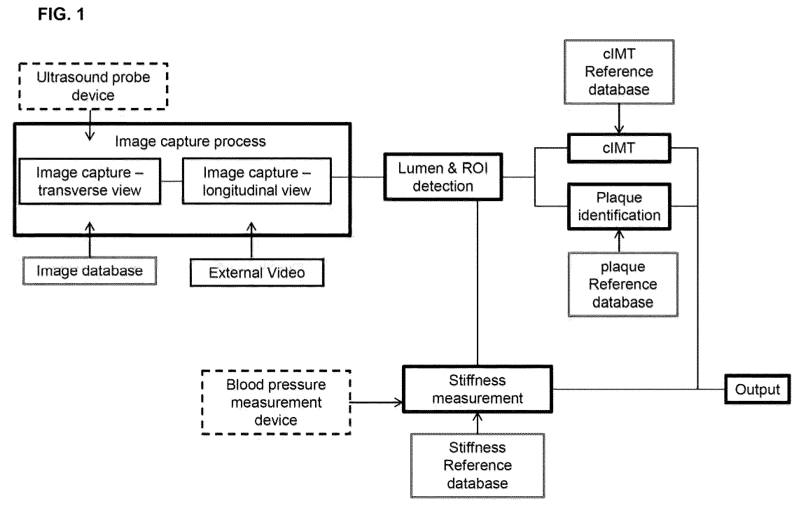

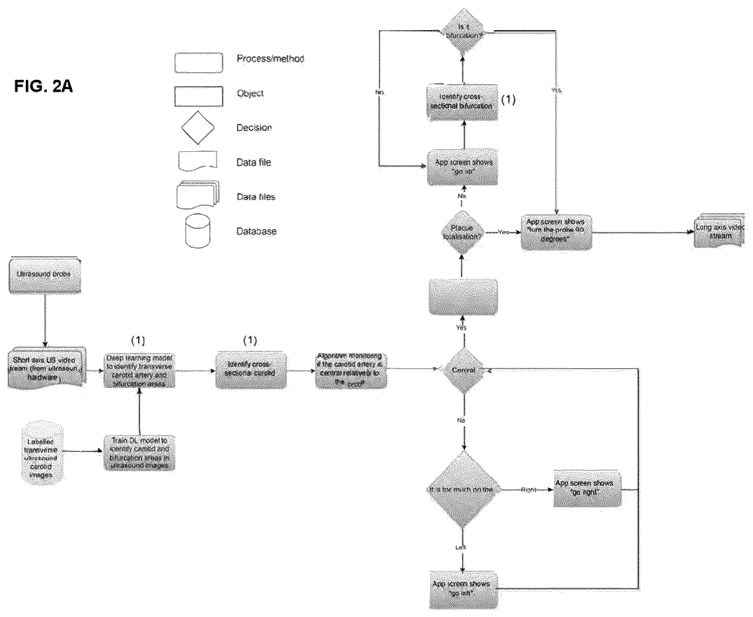

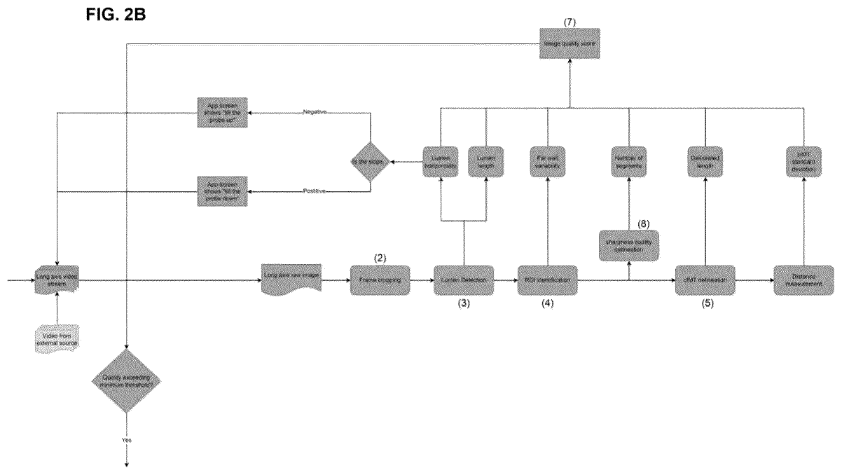

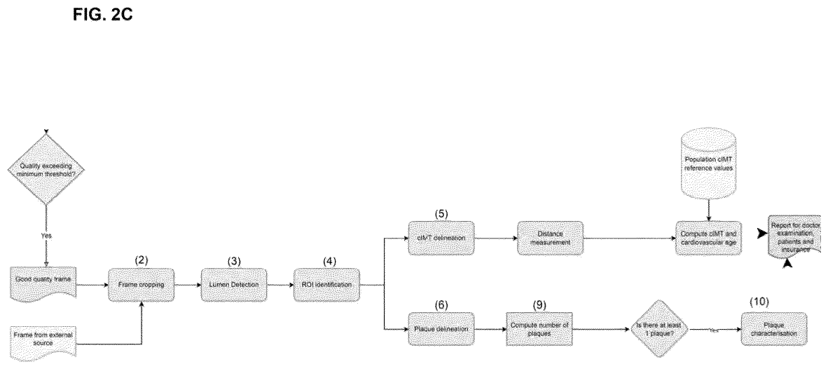

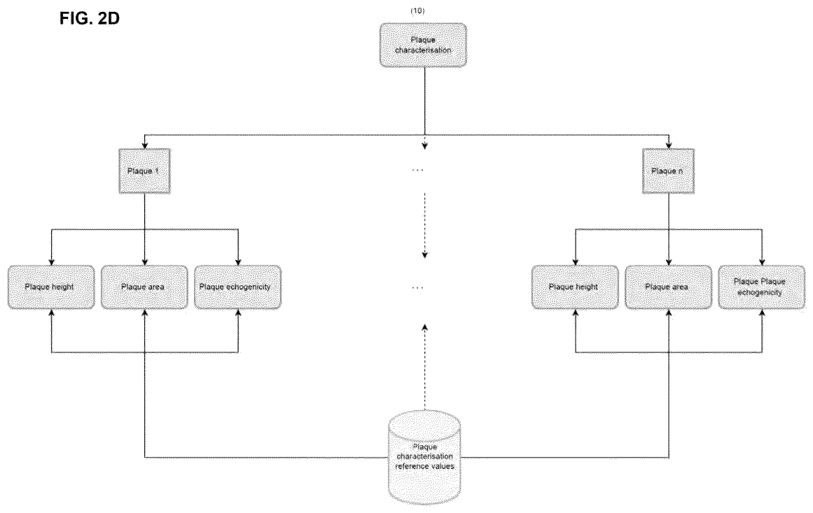

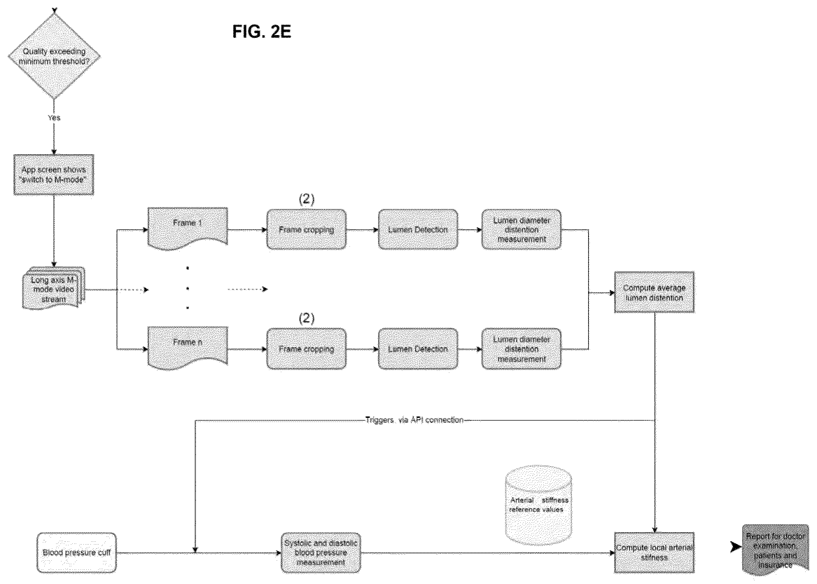

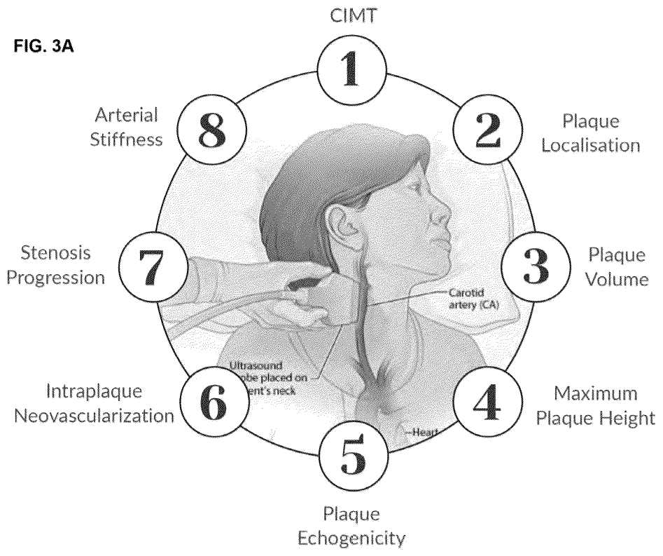

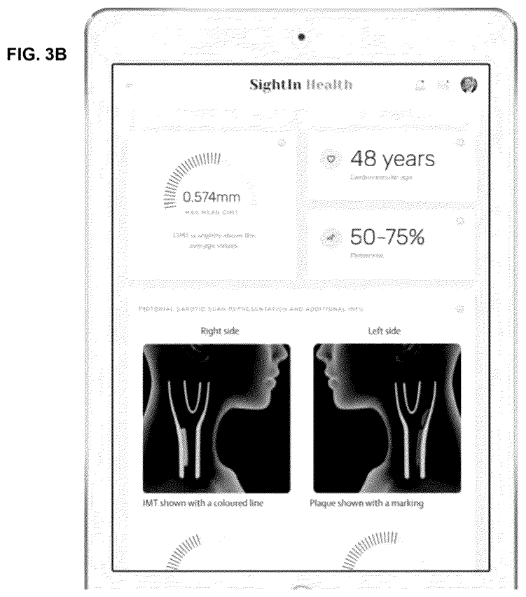

Referring to the Figures, starting in particular with FIG. 1 and FIGS. 2A to 2E, a B-mode ultrasound image processing system for measurement of cardiovascular parameters is schematically illustrated. The processing system according to some embodiments comprises a computing system with program modules installed in the computing system. The program modules are configured to process data received from an ultrasound probe device, in particular a portable ultrasound probe device that may be a commercial off the shelf portable ultrasound device used by medical practitioners for brightness mode (B-mode) ultrasound image capture of organs in a patient's body, including of a patient's carotid artery. The data transferred by the ultrasound probe device includes in particular a video stream of the ultrasound images picked up by the ultrasound probe device during medical investigation of a patient's internal organs by the medical practitioner. Other measurement devices may be connected to the computing system, for example a blood pressure measurement device that may in particular be used to supply data on the patient's blood pressure that may be used inter alia for arterial stiffness measurement as will be described in more detail hereafter.

In one embodiment, the computing system includes a memory that may store various databases. The computing system may be connected to a communications network and configured to access various databases on external servers or on the cloud. The databases may comprise an image database of still and/or moving images that may be used as a reference database for a comparison with images captured by the medical practitioner or that may be used for training an artificial intelligence program for improving the processing of image data for more accurate measurement of cardio vascular parameters. Artificial intelligence programs are well known and need not be further described herein.

In one embodiment, the computing system may include an output device that may include for example a computer, smartphone, or smartpad with a graphical user interface to display cardiovascular health measurement parameters as illustrated for example in FIG. 38. The displayed information may be adapted for consultation of measurement results by the medical practitioner, or by the patient. The output device may be a separate device or may be integrated in a computer connected to the portable ultrasound device.

In some embodiments, various program modules installed in the computing system execute various processes for cardiovascular health parameter measurement. The program modules can include an image capture module that receives and processes incoming video streams from the ultrasound probe device or an external video source. The image capture module may be configured to display the captured ultrasound image on a screen of the computing system visible to a medical practitioner during the image capture process, whereby the image capture process module may provide feed-back to guide the medical practitioner. In particular, the image capture process may be configured to guide the medical practitioner to scan the carotid in the short axis until the carotid bifurcation is reached and then to indicate when the ultrasound probe may be reoriented to start scanning the carotid in the long axis. This allows for example to measure atherosclerotic plaque and cIMT. An example of a process of transverse carotid and bifurcation identification is described in more detail hereafter and illustrated in FIGS. 2A and 2B.

According to some embodiments, detailed examples of lumen and ROI detection are provided in the detailed examples hereafter. The process flow chart of the image capture process module illustrated as an example in FIGS. 2A and 2B also illustrates a process of lumen detection and region of interest (ROI) determination included in the image capture processing.

According to some embodiments, one feature is the manner in which the lumen and region of interest are identified and extracted for further processing to determine the cIMT, identify and characterize plaques, and measure arterial wall stiffness in conjunction with a blood pressure measurement device.

According to some embodiments, the program modules may function generally in the following manner.

A raw image taken substantially along a plane parallel to the carotid longitudinal axis, showing a longitudinal cross-section of the carotid artery captured by an ultrasound probe device, is received by the computing system and processed by the program modules. The image may also be received from an image database, or an external video. The program modules process the image for measurement of parameters relevant to the assessment of cardiovascular health.

In some embodiments, the overall process of the system may include one, selected ones, or all of the following steps:
1. Transverse Carotid and Bifurcation Identification
2. Frame Cropping
3. Lumen Delineation
4. Far Wall ROI Segmentation
5. cIMT Delineation
6. Plaque Delineation
7. Image Quality Delineation
8. Sharpness Quality Delineation
9. Plaque Identification
10. Plaque Characterization
11. Wall Tracker
12. Waveform Processor

1. Example of Transverse Carotid and Bifurcation Identification

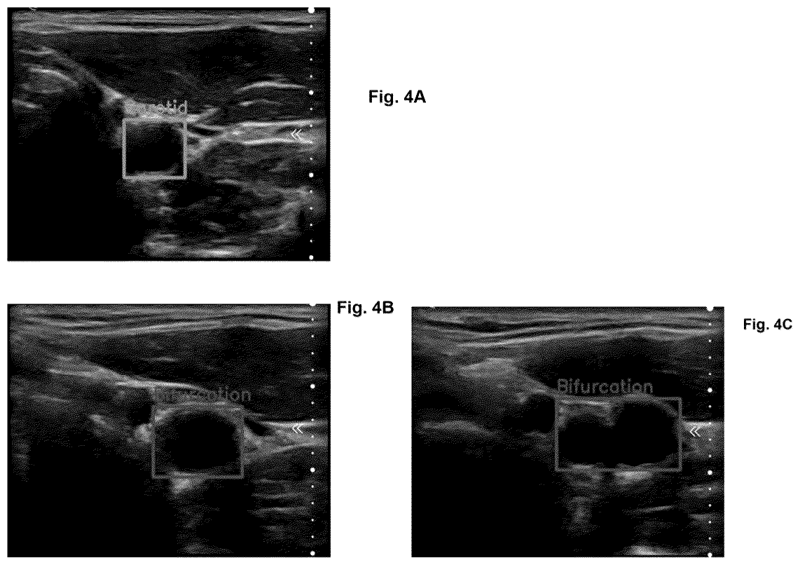

The practitioner is guided by the system to scan with the ultrasound probe device the carotid in the short axis (FIG. 4A) until the carotid bifurcation is reached (FIGS. 4B and 4C) and then is guided to turn the ultrasound probe device about 90° to start scanning in the long axis for measurement of atherosclerotic plaque and cIMT. In order to follow this process, the practitioner needs to be able to identify both the carotid lumen and bifurcation in the short axis view for which assistance is provided by the system that identifies and marks the carotid and the bifurcation.

Although there are some prior studies (see, e.g., references[1], [2]) listed at the end of the specification) regarding using Deep Learning model architectures to locate the carotid lumen structure in the short axis, such procedures have not been suggested to locate the bifurcation region as disclosed and described herein.

### Example of Identifying a Carotid Lumen

A Convolutional Neural Network (CNN) such as a YOLOv5 object identification Deep Learning model may be trained to identify the transverse view of the common carotid artery. More specifically, bounding boxes for the carotid artery in the cross-sectional view are labelled, and then the Deep Learning algorithm can be trained to learn to identify such structures. In FIG. 4A one may observe a short axis view of the carotid artery, with the corresponding bounding box. (See FIG. 4A: Short axis view of the common carotid artery, where the lumen is located inside the green bounding box.)

### Example of Identifying a Carotid Lumen Bifurcation

The method assumes as input a raw image with the carotid lumen bounding box position(s). When the carotid starts to bifurcate, the substantially circular lumen first gets deformed into an oval shape, and right after, the two lumens corresponding to the internal (ICA) and external (ECA) carotid arteries start to be distinguished. Thus, the carotid bifurcation is identified by the program module if one of the two following conditions is satisfied:
(i) The ratio of the length of the sides of the bounding box is above a certain fixed threshold. [see FIG. 4b: Image displaying the carotid bifurcation inside the red bounding box. In this image the bifurcation is identified because the bounding box of the carotid has a larger length than height.]
(ii) There are two bounding boxes for the carotid lumen that are overlapping. [see FIG. 4b: Image displaying the carotid bifurcation inside the red bounding box. In this image the bifurcation is identified because there are two overlapping bounding boxes for the carotid lumen (corresponding to the ICA and the ECA.]

### 2. Example of Frame Cropping

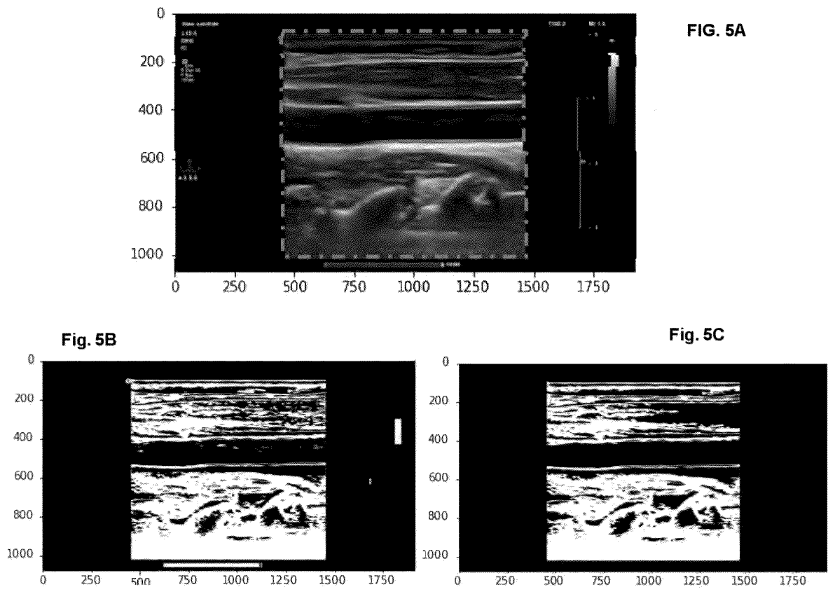

Before any analysis, B-mode ultrasound images (FIG. 5A) need to be cropped to get rid of the background/metadata information that is surrounding the image frame. [see FIG. 5A: Raw B-mode ultrasound image.]

One example of an algorithm for frame cropping may be based on a known cropping algorithm, for example as described in reference [3], with substantial modification for use in some embodiments.

In one embodiment, the frame cropping algorithm assumes the image has 3 classes of pixels:
Dark pixels, which correspond to the image background or hypoechogenic tissues like blood
Medium bright pixels, which correspond to echogenic tissues
Very bright pixels, which correspond to letters, graphical markings, or wall interfaces (very echogenic tissue)

According to some embodiments, the method may be implemented in the following steps:
The program module applies compute two thresholds, a first threshold T1, and a second threshold T2, that separate these three classes of pixels, using for example a method as described in reference [4] named herein as the Otsu method;
The program module retrieves a binary mask (see FIG. 5B), where 1's (bright pixels) corresponds to all pixels whose intensity values lie between T1 and T2 (see FIG. 5B: Binary mask where white pixels correspond to pixels with intensities between the Otsu thresholds T1, T2);
Groups of connected bright pixels with less pixels that a certain threshold are set to 0. Groups of connected bright with at least one pixel lying in the top edge of the image are also removed. FIG. 5C displays the binary mask after removing isolated pixels.

In one embodiment, the final cropped image is defined as the smallest rectangular region enclosing all remaining bright pixels. The rectangular dotted line in Figure SA represents the region of the final cropped image corresponding to the raw frame.

### 3. Example of Lumen Delineation

In carotid ultrasound images, interesting biomarkers such as cIMT, or atherosclerotic plaque are usually measured at the far wall, which is the artery wall located adjacent the lumen, on the far side of the lumen with respect to the ultrasound probe device that captures the image.

For ease of reference, it may be noted that in all of the illustrated brightness mode ultrasound images in the appended Figures, the tissue closest to the ultrasound probe is at the top of the image and the tissue furthest from the ultrasound probe device is at the bottom of the image.

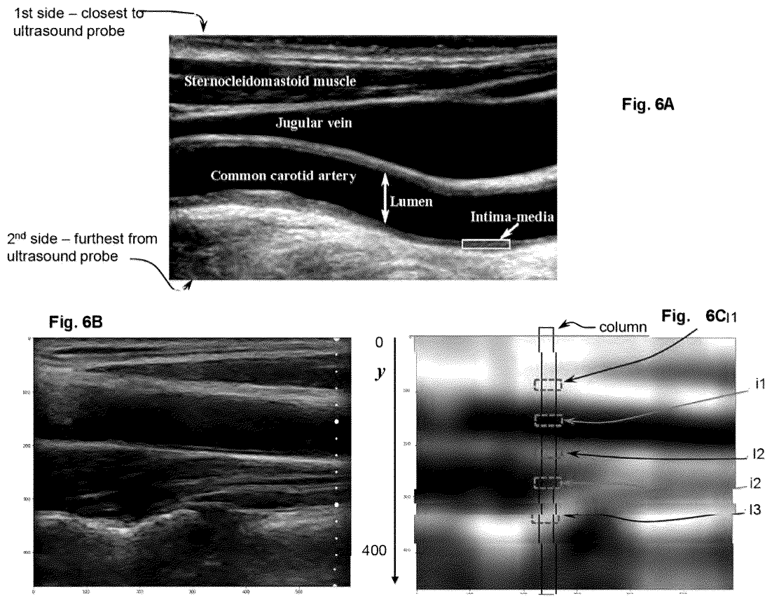

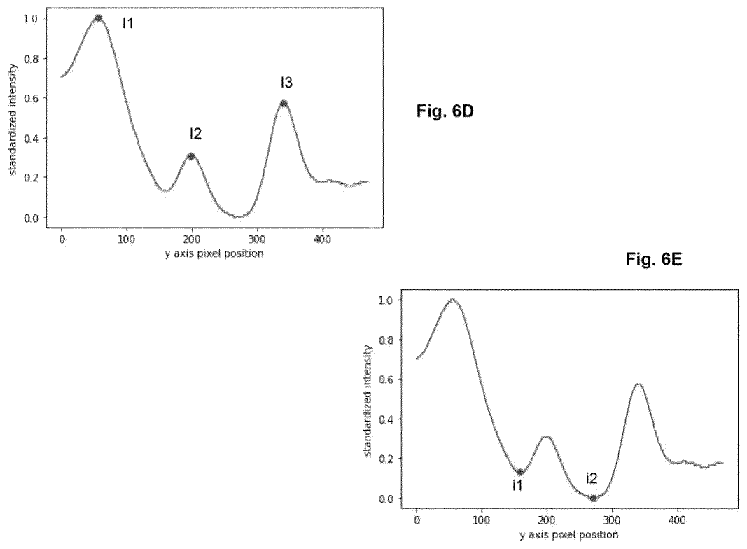

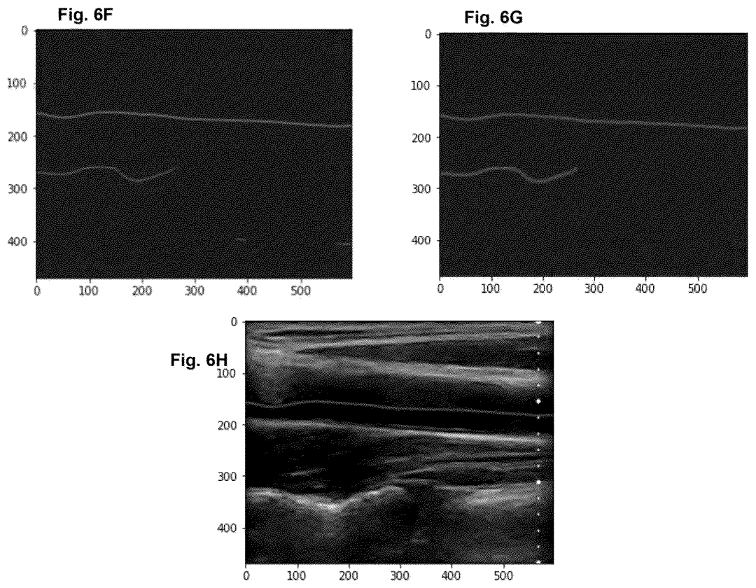

The identification and localization of the lumen structure is as such a common task in ultrasound image analysis for carotid artery analysis (see FIG. 6A: Ultrasound image of the longitudinal view of the carotid; see also Reference [5]—“Robust common carotid artery lumen detection in B-mode ultrasound images using local phase symmetry.”) The general idea of locating the lumen as the minima of a smoothed version of a cropped image is also known from references [5], [6], and [7].

In prior known methods, the correct location of the lumen is however often difficult or prone to error and moreover does not identify an optimal region of interest for the subsequent analysis of cIMT.

According to one embodiment, therefore, the system enables accurate automated identification and delineation of the lumen axis and identification of a region of interest (ROI) in the far wall for an accurate and reliable subsequent automated assessment of cIMT and optionally other cardiovascular health parameters such as plaque identification and arterial stiffness analysis.

In one embodiment, the algorithm of the program module for lumen delineation is configured to locate the lumen axis and to allow for a subsequent selection of a high-quality region of interest. The algorithm flow may thus include:
The cropped image is first smoothed using a smoothing filter, for example a 2D Gaussian image smoothing filter. [see, e.g., FIG. 6B: Raw image and FIG. 6c gaussian-filtered image.]
The resulting pixel intensity values may advantageously be standardized by scaling the values within a reference frame, for example scaled to values lying between 0 to 1.

The image may also be divided in a plurality of columns for processing column by column, so for each column pixel brightness intensity values lie for example between 0 and 1. A column may comprise a plurality of pixels in width, for example in a range between 3 to 100 pixels, depending on the chosen resolution, whereby the pixel intensity of a row of pixels across the column width is computed for example as a mean value. Pixel brightness intensity values for a given column of the smoothed image are illustrated by way of example in the plots of FIGS. 6D and 6E. In these plots, the y axis is the vertical axis and depicts the pixel intensity going from the top (positioned closer to the ultrasound probe device) to the bottom (positioned further from the ultrasound probe device) of the cropped and smoothed image.

Then, and in one embodiment, for each column, the lumen axis candidates may be identified by the program module as follows:
The significant local intensity maxima I1, I2, I3 are identified (e.g., by computing the derivatives of the plot to find the peaks) and retaining maxima that exceed a fixed intensity value threshold, for example a fixed intensity threshold having a value between 0.1 and 0.3, for example set at 0.2. FIG. 6D represents the intensity profile of a 10th column of FIG. 6c by way of example. Three local maxima are identified and all of them are above a chosen fixed threshold of 0.2 (see FIG. 6D: Standardized intensity profile with local significant maxima flagged with red dots.)

After identification of the significant local intensity maxima I1, I2, I3, between rows defined by the first and the last significant intensity maxima I1, I3, two smallest significant local intensity minima i1, i2 (see FIG. 6E, where standardized intensity profile with lumen candidates are flagged with green dots), are identified (e.g., by computing the derivatives of the plot to find the troughs). The computed two smallest significant minima i1, i2, between the first and the last significant intensity maxima, are retained as lumen candidates. The choice of two candidates instead of one is made to avoid missing the actual lumen in images where the jugular vein, or other dark structures are also present.
A binary mask where 1's corresponds to the lumen candidate positions found in the previous step is generated. [see FIG. 6f: Binary mask where 1's (blue dots) corresponds to lumen axis candidate positions]
The program module sets to 0 all isolated 1's pixels in the binary mask and then groups all bright pixels into separate lumen axis candidates' paths.
From left to right, the program module selects the first column c_i1 with any bright pixels, and initializes lumen paths as s_1=[p_i1,j1], . . . , s_n=[p_in,jn], where p_i,j are the coordinates of the bright pixels in column c_i
For each subsequent column ik:
For each bright pixel p_ik,jl, the y axis distance between p_ik, jl and all the end-points s_1[−1], . . . , s_n[−1] is computed. If the minimum distance of all pairs is below a certain threshold, the program module appends p_ik, jl to the path s*=argmin(distance(p_ik,ji, s_m)) for m=1, . . . n.
If such vertical distance is above a certain fixed threshold, this lumen candidate is considered as the beginning of a new path s_n+1=[p_ik,jl]. See FIG. 6G: Image representing in different colors 3 different lumen axis paths candidates.

According to one embodiment, all paths whose length is above a chosen proportion x times the longest path length are retrieved, and the lumen is identified as the path furthest from the ultrasound probe device (bottom-most path in the illustrated images). By selecting a conservative threshold, for example of x above 0.8, for example x=0.9, the final lumen candidate is the green line represented in FIG. 6h, where a raw image with the final lumen axis choice is displayed in green.

### 4. Example of Far Wall ROI Segmentation

Both the cIMT and atherosclerotic plaques are measured at the far wall region of the common carotid artery, which is what is defined herein as the selected region of interest (ROI). Thus segmentation of the far wall is a step needed before delineating the intima-media and media-adventitia interfaces. As the far wall is located below the lumen, the selected far wall identification method requires the lumen axis position as an input parameter.

Although the idea of defining the ROI as an envelope around the media-adventitia interface is per se known from reference [8], the optimum selection of the ROI based on the lumen delineation as described above and the longest connected path selection as described below according to an advantageous embodiment was not previously known and allows to select an optimal ROI for accurate and reliable cIMT calculation.

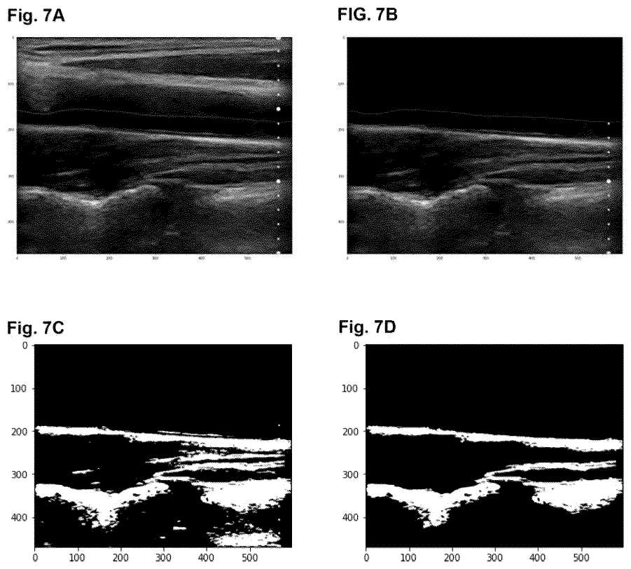

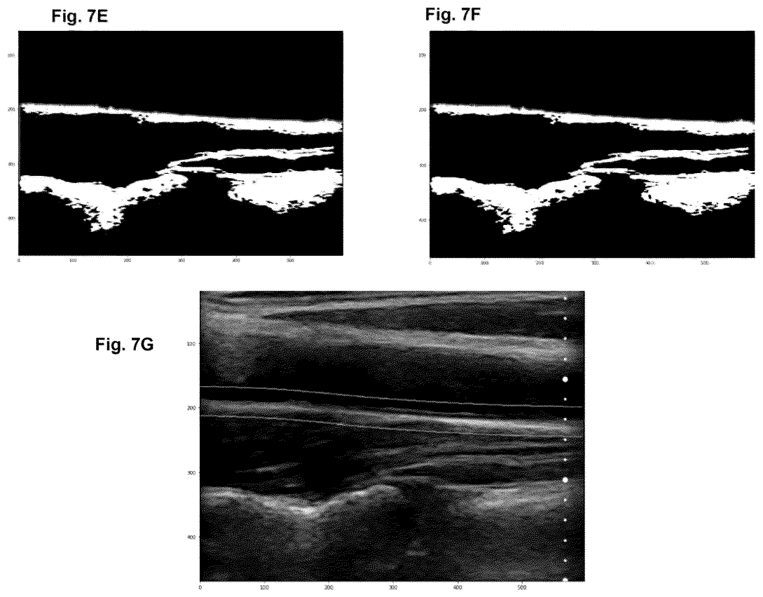

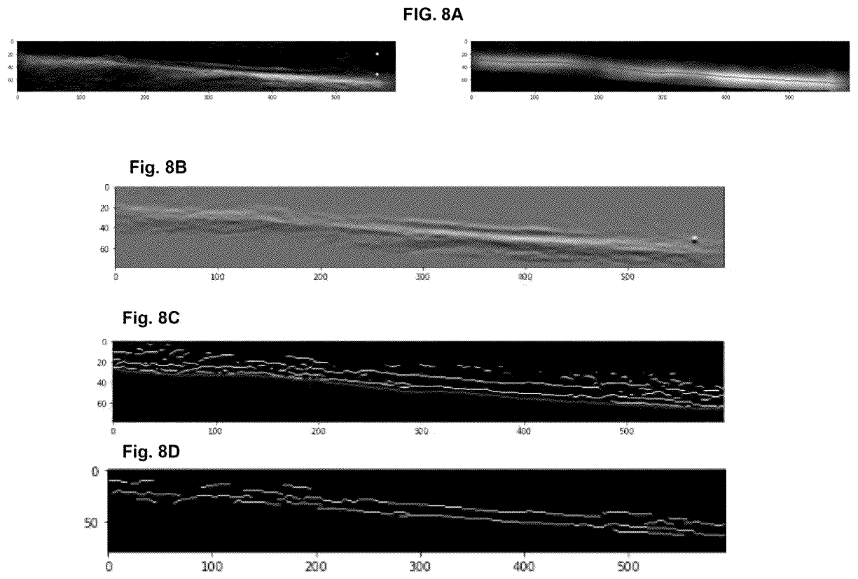

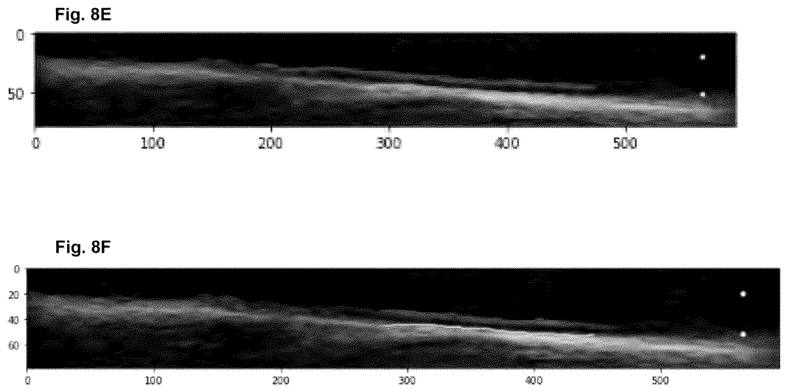

According to one embodiment, the flow of the ROI segmentation algorithm may be as follows:
The intensity of pixels above the lumen is set to 0. See FIG. 7A, where a raw image with a lumen is displayed in green and FIG. 7B, the resulting image after setting to 0 all pixels above the lumen.
A threshold T1 using an Otsu thresholding method is computed to separate pixels into two classes according to their brightness:
   Dark pixels corresponding to the lumen, all pixels above it, and other hypoechoic tissues
   Bright pixels corresponding to the wall interfaces below the lumen and other hyperechoic tissues.
A binary mask setting to 1's all pixels above the Otsu threshold T1 is computed. See FIG. 8C: Binary mask where bright pixels correspond to all pixels whose intensity is above the Otsu threshold T1].
A new binary mask with all large connected components is retrieved. A connected component is defined as “large” if the number of connected pixels is above a selected proportion of the number of connected pixels of the largest connected component. See FIG. 7D: Binary mask corresponding to the largest bright connected components. The selected proportion is preferably in a range of 0.5 to 1, preferably 0.7 to 1.
For each column, the far wall vertical position y is identified as the first bright pixel (from top to bottom in the image of the FIG. 7E) of the binary mask. See FIG. 7E: Binary mask with the first estimate of the far wall displayed in red.
Once the estimated the vertical y coordinates array for the far wall are identified, the final estimate is defined as the largest sub-segment not containing any NaNs or jumps exceeding a fixed threshold. In FIG. 7F one may observe how the green line corresponding to the final far wall estimate is not containing the initial jump of the red line in FIG. 7E, where a binary mask with the final estimate of the far wall is displayed in green.
Finally, the far wall array may be smoothed with smoothing filter, for example a Savitzky-Golay filter, and the upper and lower bounds of the far wall ROI may be defined with a predefined height parameter falling within a preferred range. In FIG. 7F, the green line corresponds to the smoothed far wall and the orange lines correspond to the lower and upper bounds of the ROI.
   5. Example of cIMT Delineation

The cIMT is defined as the distance between the lumen-intima interface and the media-adventitia interface, and it is typically measured at the far wall of the common carotid artery.

Some aspects relating to locating the adventitia as the brightest line and then restricting the lumen-intima and media-adventitia search above that line are discussed in reference [10], and some aspects of lumen-intima and media-adventitia as parallel continuous lines along maximum gradient paths are described in reference [11].

However, according to some embodiments described and disclosed herein, the cIMT delineation algorithm of the program modules performs the cIMT delineation in the provided far wall region of interest. In one embodiment, the algorithm flow is as described below.

The cIMT delineation algorithm applies a filter, for example a gaussian filter, on the far wall ROI image to reduce speckle noise. We refer to the resulting image as the smoothed intensity map I.

The center of the adventitia layer is retrieved as the continuous path passing through maximum intensity values. In other words, the adventitia layer is retrieved as the path that maximizes the score map defined as C(x, y)=max(C(x−1,y−1), C(x−1,y), C(x−1,y+1))+I(x,y), where I(x,y) are the values of the smoothed intensity map. The score map and the path optimization may be computed with a Dynamical Programming (DP) approach so that the computational cost is minimal. See FIG. 8A: Far wall ROI raw image (left), and corresponding (Gaussian) smoothed intensity map (left) with the center of the estimated adventitia displayed as a red line.

A Sobel filter may be applied in the vertical direction y to retrieve the y-gradient map of the region of interest. See FIG. 8B: y-gradient map of the far wall ROI obtained by applying a Sobel filter on the raw image.

In one embodiment, the program module retrieves all the gradient local maxima located above the adventitia center. Indeed, one is looking for the media-adventitia, and lumen-intima edges, so one should discard all edges after the adventitia. See FIG. 8C: Binary mask where 1's (white pixels) corresponds to gradient local maxima in the y axis direction, located above the adventitia center. The adventitia center is displayed as a red continuous line.

The program module selects a pre-defined number of the largest connected components, for example the 20 largest, which are considered as possible lumen-intima or media-adventitia candidates. The number 20 is arbitrary but for example may be based on a value selected by a programmer after visual inspection and validation of several images. See FIG. 8D: Largest 20 connected components that correspond to edges.

In one embodiment, the program module may retrieve the lumen-intima and media-adventitia paths as follows:
If the two largest connected components c1, c2 have a significant overlap (above a fixed threshold, in our case 0.3 times the largest connected component length) in the y axis, they are selected as final candidates. Otherwise, the overlaps between c1, c2 are computed and all the other largest connected components c3, . . . , c20. The program module then selects as final candidates the pair with the largest overlap. From the final pair of candidates, the uppermost path is identified as lumen-intima, and the lowermost path is identified as the media-adventitia

If the two largest connected components c1, c2 have a significant overlap (above a fixed threshold, in our case 0.3 times the largest connected component length) in the y axis, they are selected as final candidates. Otherwise, the overlaps between c1, c2 are computed and all the other largest connected components c3, . . . , c20. The program module then selects as final candidates the pair with the largest overlap. See FIG. 8E: Final lumen-intima (red) and media-adventitia (yellow) candidates.

According to one embodiment, the final lumen-intima and media-adventitia delineations can be seen in FIG. 8F, and are given as the delineations in the columns where both lumen-intima and media-adventitia are identified. See FIG. 8F: Final lumen-intima (red) and media-adventitia (yellow) delineations.

### 6. Example of Plaque Delineation

According to some embodiments, the aim of this method is to provide a delineation throughout the whole ultrasound image so that if there is any plaque it can be identified. In contrast with the cIMT delineation, where we are just interested in delineating a small region where we can accurately measure the cIMT, delineation towards plaque identification is run throughout the whole image.

The idea of locating the media-adventitia as a path that maximizes the gradient was already introduced in reference [12]. Another fully-automatic plaque delineation can be found in reference [13].

However, and according to some embodiments, the algorithm flow can be divided into 3 stages:

### Example of Lumen-Intima Initial Delineation

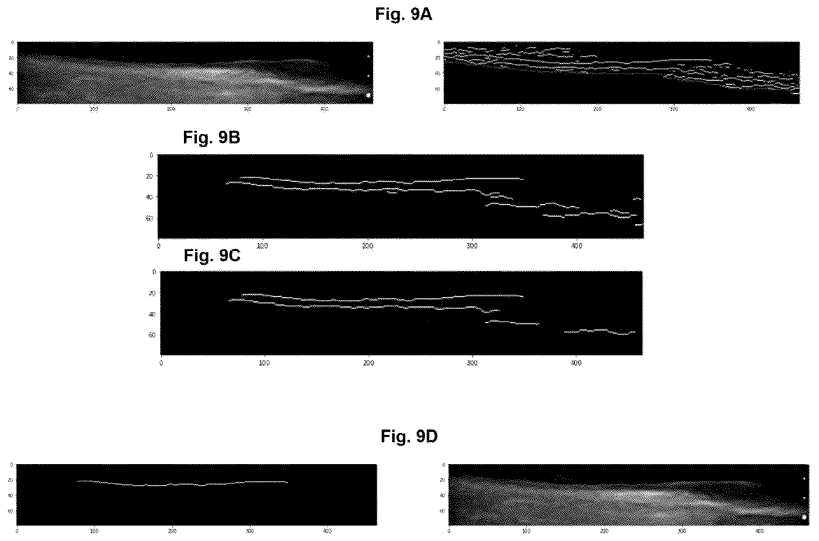

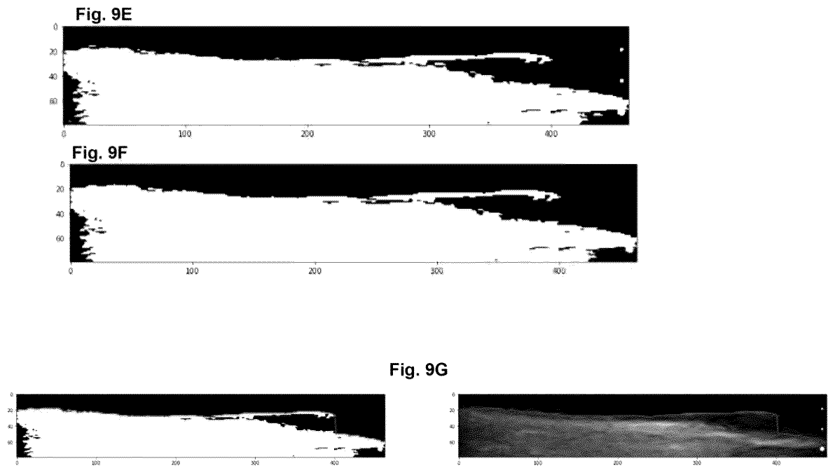

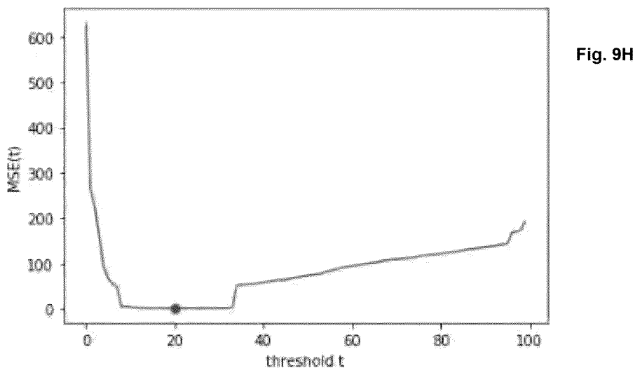

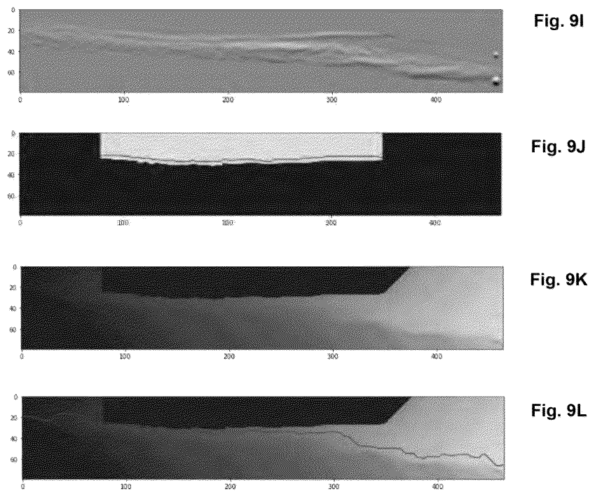

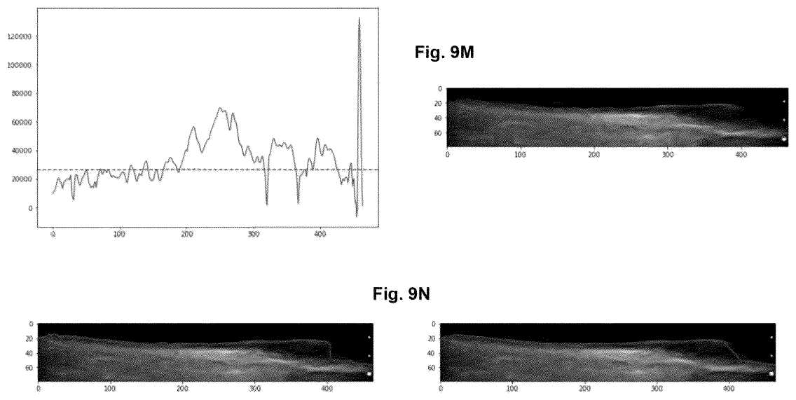

According to one embodiment, the flow of this first stage is the following: Follow steps 1-4 of the cIMT method to obtain all the gradient local maxima located above the adventitia center. See FIG. 9A: Raw far wall ROI image (left) and binary mask (right) where 1's (white pixels) correspond to gradient local maxima in the y axis direction, located above the adventitia center. The adventitia center is displayed as a red continuous line.

Connected components or paths where all pixels have gradient values below a certain fixed threshold are considered as weak edges and set to 0 by the program module. See FIG. 9B: Binary mask where 1's (bright pixels) represents strong edges.

The program module further sets to 0 all edges whose length is below a certain fixed threshold. See FIG. 9C: binary mask where 1's (bright pixels) represent long and strong edges.

By definition, the lumen-intima edge is right after the lumen, and above the media-adventitia, so we should expect that there are no edges above it, but there are edges below it. Thus, and according to one embodiment, the program module selects as initial lumen-intima candidates those edges containing:
a minimum fixed % of pixels don't have any other bright pixel above.
a minimum fixed % of pixels that have other edge pixels below. [see FIG. 9d: Initial lumen-intima delineation estimate (left), and raw far wall ROI image with the superposed lumen-intima delineation in red (right).]

### Example of Lumen-Intima Final Delineation

According to one embodiment, and given the lumen-intima initial estimate computed in the steps described in the first stage above, the flow of the second stage is the following:
Given an optimal threshold T* (detailed computation shown below), the program module retrieves a binary mask with 1's corresponding to all pixels whose intensity value is above T*. See FIG. 9E: Binary mask result of thresholding the raw image with the threshold T*.
The program module retrieves the largest connected component of the mask computed in the preceding step. See FIG. 9F: Largest connected component of the binary mask computed by thresholding the raw image with T*.
The final lumen-intima delineation is computed as the array of uppermost positions of the largest connected component retrieved in the preceding step. See FIG. 9G: Largest connected component with the final lumen-intima delineation in red (left), and raw image with the final lumen-intima delineation in red (right).
In one embodiment, the computation of the optimal threshold T* used in the above three steps may be performed as follows:
For each integer threshold candidate t between 0 and a certain fixed maximum value:
The program module follows the above three steps to obtain a lumen_intima estimate corresponding to the threshold t. We refer to it as li(t).
The program module computes the mean squared error (MSE) between li(t) and the initial estimate of the lumen-intima interface computed in the steps of the first stage.
The program module selects the optimal T*=min_t(MSE(t)), so the threshold T* t for which the MSE was minimized. See FIG. 9H: Graph displaying the MSE against the different values of the threshold t. The MSE is minimized at T*=20.

### Example of Media—Adventitia Estimation

According to one embodiment, the program module computes the far wall ROI y gradient map by applying a Sobel filter to the ROI image and denotes the gradient map. See FIG. 9I: y gradient map after applying a Sobel filter to the raw far wall ROI image.

Before computing the score map, the program module computes the lumen-intimal basin mask, which we will refer to as B. Given the lumen-intima initial estimate computed in steps of the first stage, for each column:

The program module fills with is all pixels from the top to the first gradient local maxima after the lumen-intima position, and with 0's elsewhere. See FIG. 9J Binary mask where 1's represents the lumen-intima basin. The red line represents the lumen-intima initial estimate.

In one embodiment, the program module computes a score map as follows:

| | |
|---|---|
| | C(x, y) = G(x, y)(1 + W- * (G(x, y) < 0) + min (C(x − 1, y + 1), |
| {open oversize brace} | C(x − 1, y), C(x − 1, y − 1))if B(x, y) = 1 |
| | 0 else |

See FIG. 9K: Score map C(x,y).

The program module retrieves the optimal path (path that maximizes the score along the map) by backtracking the increasing values in the score map. Note that between consecutive columns or pixels a maximum jump of 1 pixel in the y direction is allowed, so that this is a strong “smooth” condition for the selected path. The optimal path will correspond to the media-adventitia interface. See FIG. 9L: Score map C(x,y) with the optimal path corresponding to the media-adventitia display as a red line.

The program module cleans the media-adventitia by removing the sides that have low quality. More specifically, the program module keeps the segment from the first to the last pixel whose gradient value is above a proportion of the gradient maxima along the media-adventitia. See FIG. 9M: Left: gradient profile of the media-adventitia pixels. In red a threshold of 0.2 times the global maxima is displayed. Right: raw image with the final media-adventitia delineation displayed in red.

Finally, the program module smooths both lumen-intima and media-adventitia delineations using a smoothing filter, for example a Savitzky-Golay filter. See FIG. 9N: Left: Final lumen-intima and media-adventitia delineations for plaque. Right: Final smoothed lumen-intima and media-adventitia delineations for plaque.

### 7. Example of Image Quality Scoring

Image quality is important for a good cIMT delineation and for good reproducibility of results. Therefore, in some embodiment, a quality scoring system to quantify the quality of the image and ensure the system performs an analysis with good quality frames, is provided.

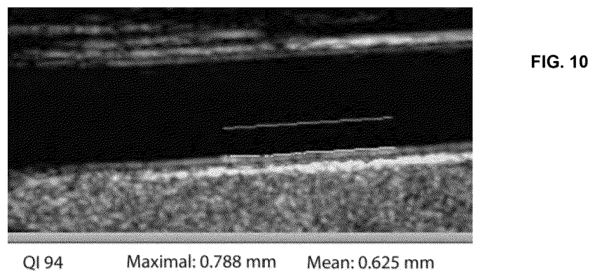

A quality index is also displayed by the Philipps QLab software, as we can see in FIG. 10, where there is shown an image taken from Philipps QLab software, and where QI=94 denotes that 94% of the points in the desired area were successfully delineated.

According to some embodiments, a quality guidance scoring system (score in the range 0-100) may, by way of non-limiting example, be provided as follows:
Lumen identification: from 0 to 15 based on the percentage of lumen that is detected in the image (linear).
Lumen horizontality: from 0 to 5 based on the angle (5 is perfectly horizontal, 0 if slope >15%, values in between follow a linear relation).
Variability in far wall: from 0 to 10 based on how smooth the far wall is (10 is perfectly smooth, 0 if Mean Square Error (original-smoothed)>35, values in between follow a linear relation).
Number of cutoff pieces of the sharpness quality delineation*: 0 to 30 (30 is 1, 0 if nb_pieces>100, values in between follow a linear relation).
Variability of the cIMT delineation: 0 to between about 5 and about 10 for the lumen-intima and about 5 for the media-adventitia. (5 if perfectly smooth, 0 if MSE (original-smoothed)>35, values in between follow a linear relation).
Length of the cimt delineation: 0 to 30: 30 if the whole width of the image has been delineated, 0 if less than 1 cm (recommended minimum length by Manheim Consensus guidelines), values in between follow a linear relation.
Thresholds to comply as best image:
If lumen identification percentage <0.8, reject.
If absolute lumen horizontality (slope)>0.15, reject, If variability in far wall (MSE)>35, reject.
If delineation length <1 cm, reject.
If the image passes all these thresholds one can consider the image as candidate for best image, and among these, the one with the highest score will win.

### 8. Example of Sharpness Quality Delineation

Image quality is critical for a good cIMT delineation and for good reproducibility of results. The program modules may include a quality scoring system to quantify the quality of the image and ensure that the systems performs analysis with good quality frames.

According to some embodiments, one goal of such an algorithm is to delineate the lumen-intima and media-adventitia interfaces to provide an additional quality metric to our quality scoring system. See reference [14].

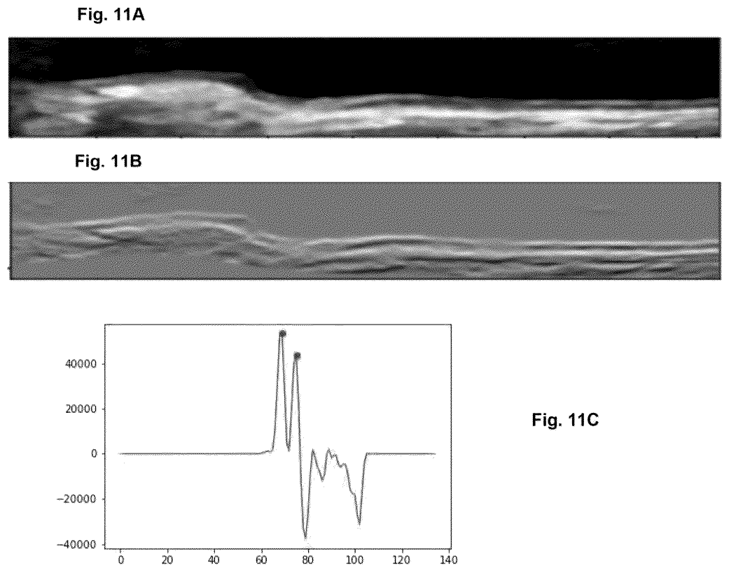

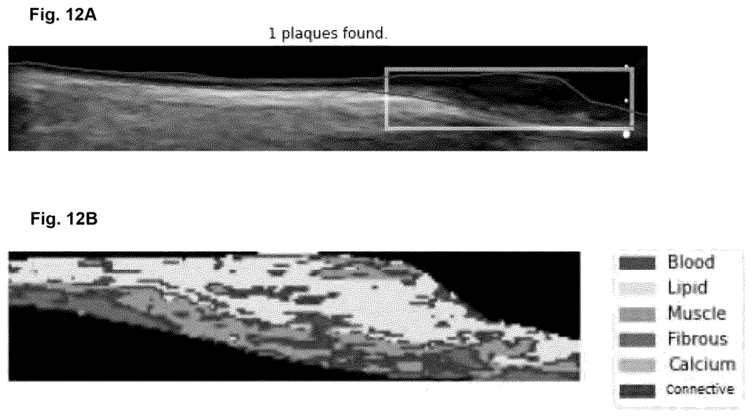

Referring now to FIG. 11A (Far wall ROI image), given a raw far wall ROI image like the one shown in FIG. 1, in one embodiment the algorithm works as follows:

The program module computes a gaussian-smoothed version of the raw image, and the y gradient map of the raw image (e.g., using a Sobel filter for the latter; see FIG. 11B: Gradient map of the far wall ROI image).

For each column of the ROI:

The program module retrieves the profile of the y-gradient image, and the profile of the gaussian-smoothed version of the grayscale pixel intensity.

The program module locates the adventitia layer position as the maximum intensity point of the smoothed grayscale profile.

The program module identifies the two biggest significant peaks (local maxima) of the gradient located before the adventitia. A maximum is considered significant if its gradient is T times the maximum gradient value in the profile, where T is a fixed threshold. See FIG. 11C: Gradient profile of a fixed column, with lumen-intima and media-adventitia points marked in red.

First and second peaks are identified with the lumen-intima and media-adventitia interfaces, respectively. If just one peak or no peaks are found the delineation for that column will be missing.

Finally, the program module retrieves the number of delineated pieces nb_pieces as the number of regions separated by missing delineated columns. Indeed, in a perfect quality image one would expect to see the lumen-intima and media-adventitia as two very sharp (high gradient) lines running along the image.

### 9. Example of Plaque Identification

The Manheim consensus (see reference [1] definition of plaque is the following:
A focal structure that encroaches into the arterial lumen of at least 0.5 mm or 50% of the surrounding IMT value or demonstrates a thickness >1.5 mm as measured from the media-adventitia interface to the intima-lumen interface.

As described in the prior art, researchers have typically used the second definition given by the Manheim consensus to identify plaque if a cIMT-delineation region exceeds 1.5 mm.

According to some embodiments, and given the raw far wall ROI image along with the delineated cIMT, the plaque location algorithm of the program modules may operate as follows;

The program module computes a median polyline distance between the lumen-intima and media adventitia, and uses such distance as baseline cIMT. It may be noted that the baseline cIMT is computed throughout the whole image except in the plaque region.

For each column, the program module computes the vertical distance between the lumen-intima point and the media-adventitia point thus obtaining a cIMT distance profile along the image.

All columns where the distance is above 1.5 mm or above 150% of the baseline cIMT are labelled as plaque-region candidates. For each set of consecutive set of plaque-region candidates:

The program module computes the median cIMT in a window after and before the region. The minimum of both measures is defined as the neighboring cIMT.

If the maximum cIMT in the plaque-region candidate is below 150% of neighboring cIMT, the region is discarded as plaque candidate.

The program module merges all remaining plaque-region candidates that are closer than a defined distance, for example 2 mm, and discards all plaque-region candidates whose length is lower than a second defined distance, for example 1.5 mm

Finally for each plaque region, plaque pixels are those lying between the delineated lumen-intima and media-adventitia interfaces.

### 10. Example of Plaque Characterization

Plaque characterization is crucial for diagnosing cardiovascular diseases. Thus, once a plaque region is identified, one can retrieve several metrics that can eventually be used for cardiovascular risk assessment or stratification.

Note that for a full plaque characterization, and according to some embodiments, we require as input parameters the mean grayscale value of the identified lumen, and the mean grayscale value of the center of the adventitia layer. According to one embodiment, the retrieved metrics are the following, and with reference to FIG. 12A (Far wall ROI raw image with the cIMT delineation in dark green and the plaque region located inside the light green bounding box:
Plaque length: the length of the plaque region is computed
Plaque height the maximum height of the plaque region is computed
Plaque area: the number of pixels that lie inside the delineated plaque
region Plaque composition is computed:

According to some embodiments, the program module first linearly normalizes all pixel values with two references: the lumen grayscale median should correspond to 0 and the adventitia grayscale median is 180. Thus, each pixel value x is transformed to x′ as follows:

0 if x < 0
(x - I_{gsm})/(adv_{gsm} = 1 _{gsm}) * 180 if 0 <= x < adv_{gsm}
180 + ((x - adv_{gsm})/(255 - adv_{gsm})) * (255 - 180) if x >= adv_{gsm}0

Each pixel may be classified into type of tissue (blood, lipid, muscle, fibrous, calcium or connective) according to their normalized intensity values:

Blood if 0 <= x′ <= 4
lipid if 8 <= x′ <= 26
muscle if 41 <= x′ <= 76
fibrous if 112 <= x′ <= 196 calcium if 211 <= x′ <= 255 connective
else

See also FIG. 12B, where plaque composition according to pixel grayscale intensities is shown.

### 11. and 12. Examples of Wall Tracking and Waveform Processors

Note that details and examples regarding data processing blocks 11 (Wall Tracker) and 12 (Waveform Processor) are set forth in the '387 provisional patent application, to which priority is claimed herein, and which is incorporated herein by reference in its entirety above.

### Some Examples of Image Processing and Machine Learning Tools that May be Used in the Different Data Processing Blocks and Steps Presented Herein

1. Transverse Carotid and Bifurcation identification
CNN's (Deep Learning)
YOLOv5 model (Deep Learning)
   2. Frame Cropping
Otsu Thresholding
Morphological (closing, opening) operations

### 3. Lumen Delineation

Gaussian smoothing
Min-max scaling (normalization technique)

### 4. Far Wall ROI Segmentation

Otsu Thresholding
   5. cIMT Delineation
Gaussian smoothing
Sobel filter

### 6. Plaque Delineation

Gaussian smoothing
Sobel filter

### 7. Image Quality

### 8. Sharpness Quality Delineation

Gaussian smoothing
Sobel filter

### 9. Plaque Identification

Various processing modules

### 10. Plaque Characterization

Various processing modules

### 11. Wall Tracker

Gaussian filter
Gradient of gaussian filter

### 12. Waveform Processor

Fast Fourier Transform
Band-pass FIR filter

### Some Other Filtering and Edge Detection Techniques that May be Used in the Different Data Processing Blocks and Steps Presented Herein

Canny edge detector
Laplacian edge detector
a ICOV filter
FOAM edge detector
Anisotropic filtering
Homomorphic filtering
Median filter

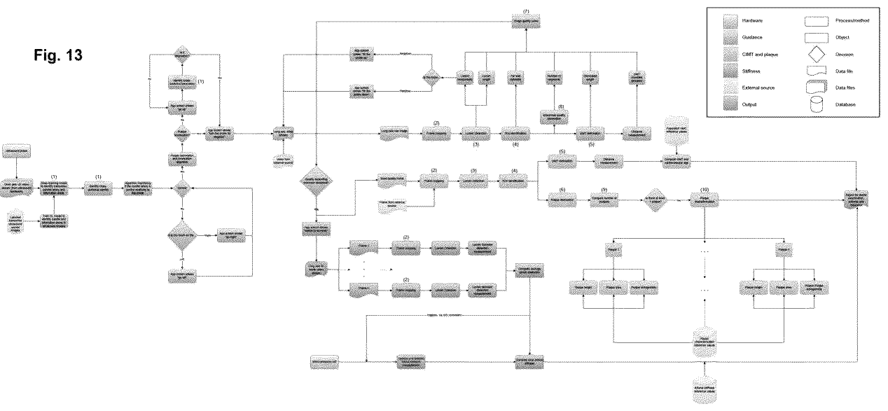

FIG. 13 shows a flow chart corresponding to another embodiment of an ultrasound image processing system for cardiovascular health assessment 3s based on ultrasound images of a patient's carotid artery according to one embodiment, aspects of which are discussed and disclosed in the specification hereof.

In accordance with some embodiments, further details regarding the specifics of the operation of the various systems, devices, components, algorithms, and methods described and disclosed above concerning data processing blocks 1 through 12 described and presented hereinabove are to be found in U.S. Provisional Patent Application Ser. No. 63/401,387 to Jatautas et al. filed on Aug. 26, 2022 and entitled “Methods, Systems, Devices and Components for Rapid On-Site Measurement, Characterization and Classification of a Patient's Carotid Artery Using a Portable Ultrasound Probe or Device,” priority to which is claimed herein, and which is incorporated by reference herein in its entirety.

Among the various systems, devices, components, algorithms and methods described and disclosed herein are various embodiments of methods and systems configured to rapidly measure, analyze and provide in, for example, an on-site and out-patient setting within a short period of time, one or more physical parameters associated with a one or more of a patient's carotid arteries. As shown above and in the Figures, some embodiments comprise at least one computing device, at least one portable handheld ultrasound device or probe operably connected to the at least one computing device, the portable handheld ultrasound device being configured to provide to the computing device as outputs therefrom a series of ultrasound image frames acquired from the one or more of the patient's carotid arteries, and a display or monitor operably connected to the at least one computing device and configured to visually display to a user results generated by the at least one computing device. Among other things, in some embodiments the ultrasound image frames can be processed to guide a clinician in accurately, quickly and efficiently placing the ultrasound probe on the patient's neck and body so that the physical characteristics of a patient's carotid arteries can be accurately detected and measured.

Described above are various embodiments of methods and systems relating to detecting and measuring various physical characteristics and properties associated with vascular systems such as the carotid arteries of a patient. Many of these embodiments work well in an environment where elongated or tubularly shaped anatomical organs such as vasculature of various types, arteries, veins, nerves and/or portions of gall bladders, are the object of the detection and measurement. However, at least some of the systems and methods described and disclosed herein may also be adapted or modified to permit the detection and measurement of various physical tot characteristics and properties of anatomical organs that are generally not elongated or tubularly shaped, such as kidneys, livers, lungs, the human heart or portions thereof, the human brain, and so on.

In other words, some of the various embodiments described and disclosed herein are not limited to carotid artery applications, and in addition to including the applications listed above in this paragraph and elsewhere herein, also find application in detecting and measuring various physical characteristics and properties associated with the femoral arteries, the aorta, the Circle of Willis in the human brain, interior and exterior carotid arteries, and so on.

Some examples of arteries that can be detected and measured according to various the embodiments disclosed and described herein include, but are not limited to, the brachiocephalic trunk artery, the subclavian artery, the common carotid artery, the external carotid artery, the internal carotid arteries, the thoracic aorta, the abdominal aorta, the iliac arteries, the axillary arteries, the brachial arteries, the ulnar artery, the radial arteries, the femoral artery, the popliteal artery, the anterior tibial artery, the posterior tibial artery, the dorsalis pedis arteries, the anterior cerebral artery, the anterior communicating artery, the middle cerebral artery, the posterior communicating artery, the posterior cerebral artery, the basilar artery, the vertebral artery, the posterior inferior cerebral artery, and any other suitable artery.

Some examples of veins that can be detected and measured according to various the embodiments disclosed and described herein include, but are not limited to, the internal jugular vein, the external jugular vein, the anterior jugular vein, the subclavian vein, the brachiocephalic vein, the superior vena cava, the azygos vein, the iliac veins, the inferior vena cava, the basilic vein, the cephalic vein, the radial vein, the ulnar vein, the brachial vein, the axillary vein, the anterior tibial vein, the posterior tibial vein, the fibular/peroneal vein, the popliteal vein, the femoral vein, the great saphenous vein, the small saphenous vein, the external iliac vein, the common iliac veins and any other suitable vein.

Additional regions of the human body that can be detected and measured that are more distantly related to the existing technology described and disclosed herein, but which would benefit from a guidance system provided in accordance with the embodiments, include, but are not limited to, bone structures, internal organs, abdominal organs, muscles, nerves, ligaments, fat tissues, connective tissues, the eyes, the brain, the skin, and pathologies such as tumors, cysts, and any other similar pathology.

Procedural guidance for syringe detection and guidance and biopsy detection and guidance can also be provided in accordance with some embodiments. In addition, the various embodiments are not limited to the use of B-mode ultrasound devices or M-mode ultrasound devices, but may also include RF-mode ultrasound devices (which can permit higher-resolution images to be acquired) and doppler-mode ultrasound devices (which, by way of example, can permit the velocity or speed of blood flow to be measured).

The data processing modules described and disclosed above may be modified individually or together to permit the use of machine learning or artificial intelligence techniques in addition to those described above.

The computing and display devices employed in the various embodiments described and disclosed herein may or may not be portable, employ cloud-based analytics and/or data transfer and distribution, be web-based, employ a cell phone or iPhone, a tablet, a laptop, or a desktop computer, operate on-line or off-line, and so on.

What have been described above are examples and embodiments of the systems, devices, components and methods described and disclosed herein. It is, of course, not possible to describe every conceivable combination of components or methodologies for purposes of describing all the various embodiments, but one of ordinary skill in the art will recognize that many further combinations and permutations of the devices and methods described and disclosed herein are possible. Accordingly, the devices and methods described and disclosed herein are intended to embrace all such alterations, modifications and variations that fall within the scope of the appended claims.

In the claims, unless otherwise indicated, the article “a” is to refer to “one or more than one.”

The foregoing outlines features of several embodiments so that those skilled in the art may better understand the detailed description set forth herein. Those skilled in the art will now understand that many different permutations, combinations and variations of the systems and methods described herein fall within the scope of the various embodiments. Those skilled in the art should appreciate that they may readily use the present disclosure as a basis for designing or modifying other processes and structures for carrying out the same purposes and/or achieving the same advantages of the embodiments introduced herein.

Those skilled in the art should also realize that such equivalent constructions do not depart from the spirit and scope of the present disclosure, and that they may make various changes, substitutions and alterations herein without departing from the spirit and scope of the present disclosure.

After having read and understood the present specification, those skilled in the art will now understand and appreciate that the various embodiments described herein provide solutions to long-standing problems in measuring and quantifying the state of health of a patient's carotid arteries or other body organs quickly, accurately, and economically.

### LITERATURE REFERENCES

[1] Automatic localization of Common Carotid Artery in ultrasound images using Deep Learning Dina A. Hassanin, Mahmoud khaled Abd-Eliah, Ashraf A. M. Khalaf and Reda R. Gharieb 2021
[2] Localization of common carotid artery transverse section in B-mode ultrasound images using faster RCNN: a deep learning approach Pankaj K. Jain. Saurabh Gupta. Amav Bhavsar. Aditya Nigam, Neeraj Sharma 2020
[3] Automatic segmentation of carotid B-mode Images using fuzzy classification Rui Rocha, Jorge Silva, Aurelio Campilho 2011
[4] A Theshold Selection Method from Gray-Level Histograms Nobuyuki Otsu 1979
[5] Automatic detection of the carotid lumen axis in B-mode ultrasound images Rui Rochaa, Jorge Silvaa, Aurélio Campilho 2013
[6] Characterization of a Completely User-Independent Algorithm for Carotid Artery Segmentation in 2-D Ultrasound Images Silvia Delsanto, Filippo Molinari, Pierangela Giustetto, William Liboni, Sergio Badalamenti, and Jasjit S. Suri 2007
[7] Automatic Computer-based Tracings (ACT) in longitudinal 2-D ultrasound images using different scanners Filippo Molinari, William Liboni, Pierangela Giustetto, Sergio Badalamenti, Jasit. S Suri 2009
[8] Intima-media thickness: setting a standard for a completely automated method of ultrasound measurement Filippo Molinari; Guang Zeng; Jasjit S. Suri 2010
[9] A Theshold Selection Method from Gray-Level Histograms Nobuyuki Otsu 1979
[10] Intima-media thickness: setting a standard for a completely automated method of ultrasound measurement Filippo Molinari; Guang Zeng; Jasjit S. Suri 2010
[11] A Fully-Automatic Method to Segment the Carotid Artery Layers in Ultrasound Imaging: Application to Quantify the Compression-Decompression Pattern of the intima-Media Complex During the Cardiac Cycle Guillaume Zahnd, Kostas Kapellas, Martijn Van Hattem, ANouk Van Dijk 2017
[12] A Fully-Automatic Method to Segment the Carotid Artery Layers in Ultrasound Imaging: Application to Quantify the Compression-Decompression Pattern of the Intima-Media Complex During the Cardiac Cycle Guillaume Zahnd, Kostas Kapellas, Martijn Van Hattem, ANouk Van Dijk 2017
[13] Segmentation of the carotid intima-media region in B-mode ultrasound images Rui Rocha, Aurelio Campilho, Jorge Silva, Elsa Azevedo, Rosa Santos 2010
[14] Real-time measurement system for the evaluation of the Intima Media Thickness with a new edge detector Francesco Faita, Vincenzo Gemignani, Elisabetta Bianchini, Chiara Giannarelli, and Marcello Demi 2006
[15] Mannheim Carotid Intima-Media Thickness and Plaque Consensus (2004-2006-2011) P. J. Touboul, M. G. Hennerici, S. Meairs, H. Adams, P. Amarenco, N. Bomstein, L. Csiba, M. Desvarieux, S. Ebrahim R. Hernandez, M. Jaff, S. Kownator, T. Naqvi, P. Prati, T. Rundek, M. Sitzer, U. Schminke, J.-C. Tardif, A. Taylor, E. Vicaut, K. S. Woo

We claim:

1. A system configured to rapidly measure, analyze and provide, in an on-site or out-patient setting and within a predetermined period of time, one or more physical parameters or characteristics associated with a one or more of a patient's carotid arteries, the system comprising:
(a) at least one computing device;
(b) at least one portable ultrasound device or probe operably connected to the at least one computing device, the portable ultrasound device being configured to provide to the computing device as outputs therefrom a series of ultrasound image frames acquired from the one or more of the patient's carotid arteries, and
(c) a display or monitor operably connected to the at least one computing device and configured to visually display to a user results generated by the at least one computing device;
wherein the computing device comprises at least one non-transitory computer readable medium configured to store instructions executable by at least one processor to process the ultrasound image frames, the at least one processor and the at least one non-transitory computer readable medium further being configured to process the ultrasound image frames using a trained discriminative or convolutional machine learning model, the computing device and ultrasound device or probe being configured to:
(i) identify at least one of a bifurcation, a transverse view, and a cross-sectional view of at least one of the patient's carotid arteries from among the ultrasound image frames generated by the ultrasound device or probe;
(ii) generate directional and locational instructions to the user on the display or monitor or via sound regarding the location, placement, orientation and movement of the ultrasound device or probe;
(iii) measure and compute at least one of lumen distention, carotid intima-media thickness (cIMT), cardiovascular age, plaque characterization, and local arterial stiffness of the at least one carotid artery; and
(iv) provide as outputs from the computing device at least one of the measured and computed lumen distention, the measured and computed carotid intima-media thickness (cIMT), the measured and computed cardiovascular age, the measured and computed plaque characterization, and the measured and computed local arterial stiffness of the at least one carotid artery to one or more of the monitor or display, a printer, a speaker or headphones, or data formatted for digital or memory storage or transmission.

2. The system of claim 1, wherein the ultrasound device or probe is configured to switchably and controllably operate in B-mode or M-mode.

3. The system of claim 2, wherein the ultrasound device or probe is configured to switchably and controllably operate in the M-mode when measuring lumen distension.

4. The system of claim 1, wherein the trained discriminative or convolutional machine learning model is trained and configured to generate directional and locational instructions to the user on the display or monitor or via sound regarding at least one of the location, placement, orientation and movement of the ultrasound device or probe to identify one or more of carotid artery bifurcation locations, carotid artery cross-sectional characteristics, carotid artery plaque characteristics, carotid artery lumen characteristics, carotid artery regions of interest, and carotid artery far wall variability.

5. The system of claim 4, wherein the trained discriminative or convolutional machine learning model is trained and configured to provide directional and locational instructions to the user regarding whether the ultrasound probe or device is centered over the patient's carotid artery.

6. The system of claim 1, wherein lumen distension is measured and computed using the patient's blood pressure as an input.

7. The system of claim 1, wherein the trained discriminative or convolutional machine learning model is trained and configured to crop the ultrasound image frames so as to enhance reliability of the detection and identification one or more of carotid artery bifurcation locations, carotid artery cross-sectional characteristics, carotid artery plaque characteristics, carotid artery lumen characteristics, carotid artery regions of interest, and carotid artery far wall variability.

8. The system of claim 1, wherein the trained discriminative or convolutional machine learning model is trained and configured to determine the quality of the ultrasound image frames so as to enhance reliability of the detection and identification one or more of carotid artery bifurcation locations, carotid artery cross-sectional characteristics, carotid artery plaque characteristics, carotid artery lumen characteristics, carotid artery regions of interest, and carotid artery far wall variability.

9. The system of claim 1, wherein the trained discriminative or convolutional machine learning model is trained and configured to measure and compute one or more of the number, location, length, height, area and echogenicity of each plaque in the at least one carotid artery as part of the plaque characterization.

10. The system of claim 1, wherein the predetermined period of time is about ten minutes or less.

11. A method of rapidly measuring, analyzing and providing, in an on-site and out-patient setting within a predetermined period of time, one or more physical parameters associated with a one or more of a patient's carotid arteries, the system comprising at least one computing device, at least one portable handheld ultrasound device or probe operably connected to the at least one computing device, the portable handheld ultrasound device being configured to provide to the computing device as outputs therefrom a series of ultrasound image frames acquired from one or more of the patient's carotid arteries, and a display or monitor operably connected to the at least one computing device and configured to visually display to a user results generated by the at least one computing device, the computing device comprising at least one non-transitory computer readable medium configured to store instructions executable by at least one processor to process the ultrasound image frames, the at least one processor and the at least one non-transitory computer readable medium further being configured to process the ultrasound image frames using a trained discriminative or convolutional machine learning model, using the computing device, ultrasound device and the display or monitor, the method comprising:
(i) identifying at least one of a bifurcation, a transverse view, and a cross-sectional view of at least one of the patient's carotid arteries from among the ultrasound image frames generated by the ultrasound device or probe;
(ii) generating directional and locational instructions to the user on the display or monitor or via sound regarding the location, placement, orientation and movement of the ultrasound device or probe;
(iii) measuring and computing at least one of lumen distention, carotid intima-media thickness (cIMT), cardiovascular age, plaque characterization, and local arterial stiffness of the at least one carotid artery; and
(iv) providing as outputs from the computing device at least one of the measured and computed lumen distention, the measured and computed carotid intima-media thickness (cIMT), the measured and computed cardiovascular age, the measured and computed plaque characterization, and the measured and computed local arterial stiffness of the at least one carotid artery to one or more of the monitor or display, a printer, a speaker or headphones, or data formatted for digital or memory storage or transmission.

12. The method of claim 11, wherein the ultrasound device or probe is configured to switchably and controllably operate in B-mode or M-mode.

13. The method of claim 12, wherein the ultrasound device or probe is configured to switchably and controllably operate in the M-mode when measuring lumen distension.

14. The method of claim 11, wherein generate directional and locational instructions to the user on the display or monitor or via sound regarding the location, placement, orientation and movement of the ultrasound device or probe to identify one or more of carotid artery bifurcation locations, carotid artery cross-sectional characteristics, carotid artery plaque characteristics, carotid artery lumen characteristics, carotid artery regions of interest, and carotid artery far wall variability.

15. The method of claim 14, wherein the trained discriminative or convolutional machine learning model is trained and configured to provide directional and locational instructions to the user regarding whether the ultrasound probe or device is centered over the patient's carotid artery.

16. The method of claim 11, wherein lumen distension is measured and computed 1 using the patient's blood pressure as an input.

17. The method of claim 11, wherein the trained discriminative or convolutional machine learning model is trained and configured to crop the ultrasound image frames so as to enhance reliability of the detection and identification one or more of carotid artery bifurcation locations, carotid artery cross-sectional characteristics, carotid artery plaque characteristics, carotid artery lumen characteristics, carotid artery regions of interest, and carotid artery far wall variability.

18. The method of claim 11, wherein the trained discriminative or convolutional machine learning model is trained and configured to determine the quality of the ultrasound image frames so as to enhance reliability of the detection and identification one or more of carotid artery bifurcation locations, carotid artery cross-sectional characteristics, carotid artery plaque characteristics, carotid artery lumen characteristics, carotid artery regions of interest, and carotid artery far wall variability.

19. The method of claim 11, wherein the trained discriminative or convolutional machine learning model is trained and configured to measure and compute one or more of the number, location, length, height, area and echogenicity of each plaque in the at least one carotid artery as part of the plaque characterization.

20. The method of claim 11, wherein the predetermined period of time is about ten minutes or less.

21. A system for processing brightness mode ultrasound images in medical applications configured for measurement of cardiovascular parameters, the system comprising a computing system and program modules executable in the computing system configured to process data generated by a B-mode ultrasound probe device of a patient's carotid artery, the program modules include modules for processing images generated by the a B-mode ultrasound probe device or probe, the device or probe further being configured to provide automated frame cropping of the images, lumen delineation, far wall ROI segmentation and cIMT delineation, wherein the lumen delineation module is configured to:
divide the image in a plurality of columns extending from a first side of the image captured closest to the ultrasound probe device to a second opposite side of the image captured furthest from the ultrasound probe device;
determine in each column at least a first and a last significant local intensity maxima I1, I2, I3 of image pixels above a chosen fixed intensity threshold;
determine in each column at least one significant local intensity minima i1, i2 of image pixels between the first significant local intensity maxima I1 and the last significant local intensity maxima I3;
compute paths of connected pixels from the local significant local intensity minima i1, i2 within a predefined distance from the significant local intensity maxima;
calculate the lengths of the paths;
select paths having a length greater than a fixed proportion of a path with the greatest length, the fixed proportion ranging between about 0.7 and about 0.95, and
define a lumen axis as the selected path furthest from the first side of the image.

22. The system according to the claim 21, wherein the intensity value of image pixels are normalized according to a fixed scale ranging between about 0 and about 1.

23. The system according to claim 22, wherein fixed intensity threshold ranges between about 0.1 and about 0.3 of the fixed scale.

24. The system according to claim 21, wherein three of the significant local intensity maxima I1, I2, I3 of image pixels above a chosen fixed intensity threshold are determined, and two of the significant local intensity minima i1, i2 are determined.

25. The system according to claim 21, wherein the redefined distance from the significant local intensity maxima falls within a predetermined range.

26. The system according to claim 21, wherein the frame cropping module is configured to generate a binary mask in which pixels whose intensity values lie between a first intensity threshold T1 and a second intensity threshold T2 are set to bright, and pixels having an intensity below the first threshold and above the second threshold are set to dark:
and further wherein the frame cropping or lumen delineation module is configured to apply a smoothing filter to the binary mask for further processing by the lumen delineation module.

27. The system according to claim 21, wherein the far wall ROI segmentation and cIMT delineation modules are configured to generate a binary mask of the image after the lumen delineation processing in which a threshold T is computed to separate pixels into two classes according to their brightness intensity:
pixels having intensities below the threshold set to dark pixels and corresponding to the lumen, and
pixels having intensities above the threshold set to bright and corresponding to the wall interfaces below the lumen and other hyperechoic tissues;
and further wherein the far wall ROI segmentation and cIMT delineation modules are configured to identify a far wall line of the artery in columns extending from the first side of the image to the second opposite side of the image, as the first line of bright pixels extending laterally across the columns.

28. The system according to claim 21, wherein the far wall ROI segmentation and cIMT delineation modules are configured to smooth the far wall line with a smoothing filter, and to define upper and lower bounds of a far wall ROI with a predefined height parameter to generate a far wall ROI image.

29. The system according to claim 21, wherein the cIMT delineation module is further configured to apply a filter to the far wall ROI image to reduce speckle noise and generate a smoothed intensity map and to apply a Sobel filter on the smoothed intensity map in the column direction y to retrieve a y-gradient map of a region of interest, and the cIMT delineation module is further configured to:
retrieve continuous paths passing through maximum intensity values defined as a center of adventitia layer,
retrieve all gradient local maxima located above the adventitia center,
select a pre-defined number of the longest paths and retaining overlapping paths, which are considered as lumen-intima or media-adventitia candidates, the paths located closest to the first side being identified as lumen-intima, and the paths closest to the second side being identified as the media-adventitia, and
define as an ROI a section having a pair of paths with the largest overlap.

30. The system according to claim 21, wherein the program modules further comprise a transverse carotid and bifurcation identification module configured to provide visual feedback to assist a medical practitioner in identifying a carotid artery and a bifurcation of the carotid artery, the transverse carotid and bifurcation identification module being configured to identify and mark with a bounding box the carotid in a transverse B-mode ultrasound image, and Identify the bifurcation if a ratio of a length to height of the bounding box is above a certain fixed threshold.

31. A method of processing brightness mode ultrasound images in medical applications for measurement of cardiovascular parameters, the method comprising executing program modules in a computing system configured to process data generated by a B-mode ultrasound probe device of a patient's carotid artery, the program modules including modules for: (a) processing images generated by the B-mode ultrasound probe device; (b) automated frame cropping of the images; (c) lumen delineation; and (d) far wall ROI segmentation and cIMT delineation, wherein the lumen delineation module performs the following steps:
divide each of the images into a plurality of columns extending from a first side of each image captured closest to the ultrasound probe device to a second opposite side of the image captured furthest from the ultrasound probe device;
determine in each column at least a first and a last significant local intensity maxima I1, I2, I3 of image pixels above a chosen fixed intensity threshold;
determine in each column at least one significant local intensity minima i1, i2 of image pixels between the first significant local intensity maxima I1 and the last significant local intensity maxima I3;
compute paths of connected pixels from the local significant local intensity minima i1, i2 within a predefined distance from the significant local intensity maxima;
calculate the lengths of the paths;
select paths having a length greater than a fixed proportion of a path with the greatest length, the fixed proportion being in a range of 0.7 to 0.95, and
define a lumen axis as the selected path furthest from the first side of the image.

32. The method according to claim 31, wherein the intensity value of image pixels is normalized according to a fixed scale ranging between about 0 and about 1.

33. The method according to claim 31, wherein three of the significant local intensity maxima I1, I2, I3 of image pixels above a selected fixed intensity threshold are determined, and two of the significant local intensity minima i1, i2 are determined.

34. The method according to claim 31, wherein the predefined distance from the significant local intensity maxima falls within a predetermined range.

35. The method according to claim 31, wherein the frame cropping module is configured to generate a binary mask in which pixels whose intensity values lie between a first intensity threshold T1 and a second Intensity threshold T2 are set to bright, and wherein pixels having an intensity below the first threshold and above the second threshold are set to dark, and further wherein the frame cropping or lumen delineation module applies a smoothing filter to the binary mask for further processing by the lumen delineation module.

36. The method according to claim 31, wherein the far wall ROI segmentation and cIMT delineation modules are further configured to generate a binary mask of the images after lumen delineation processing in which a threshold T is computed to separate pixels into two classes according to their brightness intensity: (a) pixels having intensities below the threshold set to dark pixels and corresponding to the lumen, and (b) pixels having intensities above the threshold set to bright and corresponding to the wall interfaces below the lumen and other hyperechoic tissues, and further wherein the far wall ROI segmentation and cIMT delineation modules are further configured to identify a far wall line of the artery in columns extending from the first side of the images to the second opposite side of the images, as the first line of bright pixels extending laterally across the columns.

37. The method according to claim 31, wherein the far wall ROI segmentation and cIMT delineation modules are configured to smooth the far wall line with a smoothing filter, and to define upper and lower bounds of a far wall ROI with a predefined height parameter to generate far wall ROI images.

38. The method according to the preceding claim, wherein the cIMT delineation module is further configured to apply a Gaussian filter to far wall ROI images to reduce speckle noise and generate a smoothed intensity map, apply a Sobel filter to the smoothed intensity map in the column direction y to retrieve a y-gradient map of a region of interest, retrieve continuous paths passing through maximum intensity values defined as a center of adventitia layer, retrieve all gradient local maxima located above the adventitia center, select a pre-defined number of the longest paths and retaining overlapping paths determined as to be lumen-intima or media-adventitia candidates, the paths being located closest to the first side being identified as lumen-intima, and the paths closest to the second side being identified as the media-adventitia, and define as ROI a section having a pair of paths with the largest overlap.
